(19) United States
(12) Patent Application Publication
(10) Pub. No.: US 2025/0001132 A1
(43) Pub. Date: Jan. 2, 2025

(54) SOUND-GUIDED ASSESSMENT AND LOCALIZATION OF AIR LEAK AND ROBOTIC SYSTEM TO LOCATE AND REPAIR PULMONARY AIR LEAK

(71) Applicants: The Trustees of the Stevens Institute of Technology, Hoboken, NJ (US); The Trustees of Columbia University in the City of New York, New York, NY (US); Stanford University, Stanford, CA (US)

(72) Inventors: Jinho Kim, Jersey City, NJ (US); Gordana VUNJAK-NOVAKOVIC, New York, NY (US); John D. O'NEILL, New York, NY (US); Meghan PINEZICH, New York, NY (US); Brandon A. Guenthart, Palo Alto, CA (US)

(73) Assignees: The Trustees of the Stevens Institute of Technology, Hoboken, NJ (US); The Trustees of Columbia University in the City of New York, New York, NY (US); Stanford University, Stanford, CA (US)

(21) Appl. No.: 18/292,145

(22) PCT Filed: Jul. 27, 2022
(86) PCT No.: PCT/US2022/038596

§ 371 Date: Jan. 25, 2024

Related U.S. Application Data
(60) Provisional application No. 63/226,119, filed on Jul. 27, 2021.
(60) Provisional application No. 63/332,856, filed on Apr. 20, 2022.

(51) Int. Cl. A61M 25/01; A61M 25/00

(52) U.S. Cl. CPC A61M 25/0113; A61M 25/0026; A61M 2025/0166; A61M 2202/0492; A61M 2205/3306; A61M 2205/50; A61M 2209/088; A61M 2210/1039; A61M 2230/40

(57) ABSTRACT

The disclosed subject matter relates to a device, system and method for locating and repairing a pulmonary air leak in a patient.

### STATEMENT OF GOVERNMENT SUPPORT

This invention was made with government support under EB027062 awarded by the National Institutes of Health and 2143620 awarded by the National Science Foundation. The government has certain rights in the invention.

### FIELD

The disclosed subject matter relates to a device, system and method for locating and repairing a pulmonary air leak.

### BACKGROUND

Air leak (AL) is a clinical phenomenon that is associated with the leakage or escape of air from cavity which contains air into spaces that usually under normal circumstances do not have air. The terminology Air Leak Syndrome (ALS) is the presence of air leak with associated symptoms of respiratory distress. Lacour M, Caviezel C, Weder W. Schneiter D. Postoperative complications and management after lung volume reduction surgery. J Thorac Dis. 2018 August: 10 (Suppl 23): S2775-S2779; Darwiche K, Aigner C. Clinical management of lung volume reduction in end stage emphysema patients. J Thorac Dis. 2018 August; 10 (Suppl 23): S2732-S2737; Shintani Y. Funaki S, Ose N, Kawamura T, Kanzaki R, Minami M, Okumura M. Air leak pattern shown by digital chest drainage system predict prolonged air leakage after pulmonary resection for patients with lung cancer. J Thorac Dis. 2018 June; 10 (6): 3714-3721. Air containing cavities include: tracheobronchial tree.

The escape of air from an air containing cavity to a non-air containing cavity can create a condition where some vital organs in the non-air containing cavity can be compressed creating life-threatening conditions. These life-threatening conditions can be created as a result of compression of the lung or major blood vessels. When the lung or major blood vessels are flattening by the presence of air, gas exchange or blood flow can be severely compromised. Thus, there is a need for a system and method for non-invasive detection, localization and treatment of an air leak.

### SUMMARY OF THE DISCLOSED SUBJECT MATTER

In one aspect, the disclosed subject matter provides device, system and method for locating and repairing a pulmonary air leak. The device is a non-invasive air leak detection device comprising a garment having a plurality of sensors arranged in an array for collecting acoustic signals produced by a pulmonary air leak internal to the wearer of the garment. An amplifier is operatively engaged to the sensor array, and is configured to amplify the acoustic signals detected by the sensory array. An acoustic analyzer that is operatively engaged to the amplifier receives the amplified acoustic signals and is programmed with sound localization algorithms to determine the location of the air leak in the wearer's body. In one embodiment, the garment is a vest having posterior and anterior sides.

The plurality of sensors can be arranged in a 6×6 array on the posterior side of the vest or both posterior and anterior sides of the vest. In some embodiments, the garment is a chest strap. The plurality of sensors can be arranged in a 6×6 array. Other arrangements include a 3×3 array.

The acoustic analyzer is programmed to use sound localization algorithms to assess differences in time-arrival and intensity of the acoustic sounds collected by the sensor array to detect the location of an air-leak. The acoustic analyzer may also be programmed with noise filtering algorithms to isolate unique sound signals of an air leak. For example, noise from heartbeat and breathing of a wearer of the garment may be filtered out.

In another aspect, the air leak locator device is operatively engaged to a therapeutic delivery system configured to treat the air-leak with, for example, local administration of biosealant or other therapeutic. In this regard, the therapeutic delivery system comprises a steerable applicator having a longitudinal body with a proximal end and a distal end and a length therebetween. First, second, and third channels are disposed along at least a portion of the length of the longitudinal body. The first channel is configured to receive a microphone, the second channel is configured to receive an optical fiber and the third channel is configured to receive and deliver therapeutic topically to the air leak. The first and second channels can have a diameter of 1 mm, and the third channel can have a diameter of 2 mm.

In some embodiments, the therapeutic delivery system further includes a motor and motor controller operatively engaged to the steerable applicator. The steerable applicator may include an optical fiber imaging probe disposed in the third channel along the tip of the longitudinal body of the applicator for steering by visual servoing based upon information collected via the optical fiber imaging probe. The applicator may include at least first and second pulling wires along its length making it steerable by one or more motors attached to the pulling wires.

The steerable applicator can be configured to target and deliver therapeutic by optoacoustic guidance. In this regard, the steerable applicator is preloaded with therapeutic, the therapeutic being contained in the third channel of the steerable applicator. In some embodiments, the therapeutic is a biosealant composition, such as that described in PCT/US17/55549 (Publication No. WO2018067938), entitled Cell-Seeded Porous Lung Hydrogel Sealant, the contents of which are incorporated herein by reference thereto. For example, but not limitation, the bio-sealant composition may comprise an extracellular matrix hydrogel including at least a first extracellular matrix protein, an unbranched polysaccharide, or an elastic protein, and a thermogel material including a cross-linking enzyme in an amount sufficient to result in gelation of the thermogel at a temperature from about 25° C. to about 37° C.

In an embodiment, a non-invasive air leak detection device includes a garment having a plurality of sensors arranged in an array for collecting acoustic signals produced by a pulmonary air leak internal to the wearer of the garment, an amplifier operatively engaged to the sensor array, the amplifier configured to amplify the acoustic signals detected by the sensory array, and an acoustic analyzer operatively engaged to the amplifier and configured to receive the amplified acoustic signals and programmed with sound localization algorithms to determine the location of the air leak in the wearer's body. The garment may be a vest having posterior and anterior sides. In an embodiment, the plurality of sensors are arranged in a 6×6 array. For instance, the 6×6 array can be on the posterior side of the vest. Alternatively, the plurality of sensors may be arranged in a 6×6 array on the posterior and anterior sides of the vest. In certain embodiments, the garment may be a chest strap. Alternatively, the plurality of sensors may be arranged in a 3×3 array.

In certain embodiments, the acoustic analyzer is a computer programmed to use sound localization algorithms to assess differences in time-arrival and intensity of the acoustic sounds collected by the sensor array to detect location of air-leak. In another embodiment, the acoustic analyzer is programmed with noise filtering algorithms to isolate unique sound signals of an air leak. In an additional embodiment, the acoustic analyzer can be programmed to filter out ambient noise. In other embodiments, the acoustic analyzer is programmed to filter out noise from the heartbeat and other background noises of the wearer of the garment.

In some embodiments, the non-invasive air leak detection device further includes a therapeutic delivery system having a steerable applicator with a longitudinal body having a proximal end and a distal end and a length therebetween, and first, second, and third channels along at least a portion of the length of the longitudinal body, the first channel configured to receive a microphone, the second channel configured to receive an optical fiber and the third channel configured to receive and deliver therapeutic topically to the air leak. The steerable applicator can have a first channel with a diameter of 1 mm, a second channel with a diameter of 1 mm and a third channel with diameter of 2 mm. The therapeutic delivery system can further include a motor and motor controller operatively engaged to the steerable applicator, the steerable applicator including an optical fiber imaging probe disposed in the third channel along the tip of the longitudinal body of the applicator. In an embodiment, the applicator is steered by visual servoing based upon information collected via the optical fiber imaging probe. In another embodiment, the applicator includes at least first and second pulling wires along its length and the applicator is steerable by one or more motors attached to the pulling wires. In yet another embodiment, the steerable applicator is configured to target and deliver therapeutic by optoacoustic guidance. In an additional embodiment, the steerable applicator is preloaded with therapeutic, the therapeutic being contained in the third channel of the steerable applicator. The therapeutic can be a biosealant composition, which can comprise extracellular matrix hydrogel including at least a first extracellular matrix protein, an unbranched polysaccharide, or an elastic protein, and a thermogel material including a cross-linking enzyme in an amount sufficient to result in gelation of the thermogel at a temperature from about 25° C. to 37° C.

In some embodiments, a non-invasive air leak detection device can include a garment having a plurality of sensors arranged in an array for collecting acoustic signals produced by a pulmonary air leak internal to the wearer of the garment, an amplifier operatively engaged to the sensor array, the amplifier configured to amplify the acoustic signals detected by the sensory array, an acoustic analyzer operatively engaged to the amplifier and configured to receive the amplified acoustic signals and programmed with sound localization algorithms to determine the location of the air leak in the wearer's body and a therapeutic delivery system which includes a steerable applicator having a longitudinal body having a proximal end and a distal end and a length therebetween, and first, second, and third channels along at least a portion of the length of the longitudinal body, the first channel configured to receive a microphone, the second channel configured to receive an optical fiber and the third channel configured to receive and deliver therapeutic topically to the air leak.

In an embodiment, the therapeutic device is removably attached to the wearer's body at a location selected to perform a variety of functions. Such functions can include detecting and/or diagnosing the air leak, determining the severity of the air leak, locating the site of the air leak, and/or delivering a therapeutic selected to treat the air leak.

### BRIEF DESCRIPTION OF THE DRAWINGS

FIGS. 1 and 2 are schematics depicting a device and system to treat pulmonary air leak in accordance with an embodiment of the disclosed subject matter.

FIG. 1 shows an air leak locator that is a wearable vest and acoustic analyzer that detects and locates air leaks.

FIG. 2 shows a therapeutic delivery system that is a steerable robotic applicator with an optoacoustic probe that navigates, accesses, and directly delivers a therapeutic (e.g., sealant) at the site of the leak to repair the air leak.

FIG. 3 illustrates a method of use to treat a patient with pulmonary air leak in accordance with an embodiment of the disclosed subject matter. The air leak locator estimates the region of a pulmonary air leak by detecting and analyzing the unique sound generated by the air leak. After the air leak region is determined by the air leak locator, a therapeutic delivery system introduces a robotic applicator that can navigate to the site and deliver a therapeutic payload (e.g., sealant) to treat the air leak.

FIG. 4 is a schematic illustration of needle puncture and air leak induction in rat lung. S: pressure and flow sensors. Specifically, it is a patient with a pulmonary air leak wearing a locator vest with microphone sensors on (A) anterior and (B) posterior of vest. (C) microphone sensors in locator vest detect unique acoustic signals (i.e., sound waves) produced by air leak. Acoustic signals are collected by microphone sensors, amplified by an acoustic preamplifier, and conveyed by an audio interface to a computer for acoustic analysis that results in localization of the air leak.

FIG. 5 depicts a diagram of signal isolation and noise cancellation in an acoustic analyzer in accordance with an embodiment of the disclosed subject matter. In order to isolate the acoustic signal generated by the air leak, noise such as breathing, heartbeat, and ambient sounds are removed using adjustable filters and adaptive algorithms. ADC: analog-to-digital conversion.

FIGS. 6 and 7 depict sound localization algorithm in accordance with an embodiment of the disclosed subject matter.

FIG. 6 shows sound localization via intensity and arrival time detection. FT: Fourier transform. IFT: Inverse Fourier transform.

FIG. 7 shows calculation of direction of a sound source (e.g., air leak). C: speed of sound in air.

FIG. 8 is a schematic depiction of a system for therapeutic delivery in accordance with an embodiment of the disclosed subject matter. A steerable applicator utilizes optoacoustic guidance to target and deliver therapeutic payload to the site of an air leak.

FIGS. 9 and 10 depict operation of motorized steerable applicator in accordance with an embodiment of the disclosed subject matter.

FIG. 9 shows that the applicator can be steered manually using a motor controller and a joystick.

FIG. 10 shows that the applicator can be steered fully or partially automated, by using computer vision-guided motor control methods (i.e., visual servoing), where the optical information collected via an optical fiber imaging probe at the tip of an applicator can be processed to steer the applicator.

FIG. 11 depicts visual servoing for computer-assisted steering of an applicator prototype in accordance with an embodiment of the disclosed subject matter. Specifically, a schematic of vision-guided device manipulation is shown.

FIGS. 12 and 13 depict configurations of therapeutic applicator tips in accordance with an embodiment of the disclosed subject matter.

FIG. 12 shows a probe having three channels: i) for an optical fiber imaging probe (channel diameter: 1 mm), ii) for a miniaturized microphone (channel diameter: 1 mm), and iii) for therapeutic delivery (lumen diameter: 2 mm).

FIG. 13 shows the probe of FIG. 12, showing localization followed by therapeutic delivery at the site of the air leak. Fiber: optical fiber. Mic: mini-microphone. Thera: therapeutic.

FIG. 14 depicts a method of using an embodiment of the disclosed subject matter. Specifically, it is a flow diagram of treatment of an air leak, showing, respectively, the precise site of an air leak being located using a mini-microphone at the tip of the robotic applicator; therapeutic being applied to the site of the air leak by a robotic applicator; and the air leak being repaired. F: optical fiber. M: mini-microphone. T: therapeutic. Thera: therapeutic.

FIG. 15 depicts an example of optical fiber imaging to guide and monitor therapeutic delivery to injured lungs in a rat in accordance with an embodiment of the disclosed subject matter. Specifically, an optical fiber imaging probe (diameter: 500 μm) inserted through the chest wall to monitor the lungs is depicted.

FIGS. 16 and 17 depict an assessment and localization of pulmonary air leak using sound analysis. FIG. 16 is a schematic of pulmonary air leak with computer-assisted sound analysis system. Mic: microphone. F: frequency. FIG. 17 is a process flow diagram of air leak sound analysis for severity prediction and localization to improve clinical decision-making for patients with air leak.

FIG. 18 shows a setup of a pulmonary air leak sound analysis system as a schematic of a computer-assisted sound analysis system. DAQ: data acquisition system. S: pressure and flow sensors.

FIGS. 19 and 20 are a series of plots showing the correlation between airway pressure and sound pressure level in rat lungs with air leak.

FIGS. 21-30 depict an analysis of pulmonary air leak sounds in rat lungs.

FIG. 21 is a schematic of needle puncture and air leak induction in rat lung. S: pressure and flow sensors.

FIG. 22 depicts (i) Puncture wound in rat lung; (ii) Air leak induced by puncturing the lung with a needle (18-gauge or 16-gauge); and (iii) Focal puncture wound (diameter: 1.3 mm).

FIG. 23 is a photograph of setup to monitor pressure and record air leak sounds in rat lungs.

FIG. 24 is a photograph of microphone positioned above the punctured rat lung for sound recording. Mic: microphone.

FIG. 25 illustrates pressure of inhaled air (Pairway) measured at the trachea during sound recording. Insp: inspiration. Exp: expiration.

FIG. 26 illustrates a spectrogram of the air leak sound calculated from the recorded sound showing sound frequency distribution and density. Freq: frequency.

FIG. 27 illustrates A-weighted sound pressure level (SPL).

FIG. 28 illustrates a representative air leak power spectra obtained via Fourier transform.

FIG. 29 illustrates normalized amplitude of the acquired sound signal. An air leak locator including a patient with pulmonary air leak wearing the device with microphone sensors on anterior and posterior of vest in accordance with an embodiment of the disclosed subject matter.

FIG. 30 illustrates an inverse relationship between band power and frequency in air leak power spectra (p<0.001, R2=0.702). Y=−0.00231X−49.09, where X is frequency and Y is frequency band power.

FIGS. 31-40 depict an analysis of pulmonary air leak sounds in swine lung staple line failure model.

FIG. 31 is a schematic of swine wedge resection and staple line failure model to induce pulmonary air leak for sound analysis in situ. S: pressure and flow sensors.

FIG. 32 is a photograph of right middle lobe with air bubbling at site of staple line failure air leak.

FIG. 33 illustrates a radiograph of radiopaque dye leaking from the lung parenchyma at site of staple line failure air leak.

FIG. 34 illustrates pressure of inhaled air (Pairway) measured at the trachea during sound recording. Insp: inspiration. Exp: expiration. f

FIG. 35 illustrates the normalized amplitude.

FIG. 36 illustrates A-weighted sound pressure level (SPL).

FIG. 37 illustrates a spectrogram of the air leak sound calculated from the recorded sound showing sound frequency distribution and density. Freq: frequency.

FIG. 38 illustrates a spectrogram showing sound frequency distribution and density of mild air leak. Freq: frequency. VLoss: tidal volume loss. Br. breath.

FIG. 39 illustrates power spectra of air leaks of varying severity.

FIG. 40 illustrates loudness of air leak sounds of varying severity.

FIGS. 41-44 depict localization of air leak site by measurement of relative loudness.

FIG. 41 is a rat lung photograph.

FIG. 42 is a corresponding breath sound intensity matrix heat map.

FIG. 43 is a swine lung photograph.

FIG. 44 is a corresponding breath sound intensity matrix heat map. The value in each array indicates the measured loudness normalized to the maximum loudness measured at the air leak site (dotted regions). Inset: overview photographs of analyzed regions of lung.

FIG. 45 is a schematic illustrating generation of air leak sounds.

FIGS. 46 and 47 are a spectral analysis of pulmonary air leak sounds in the rat model of FIGS. 19 and 20.

FIGS. 48-50 are a spectral analysis of pulmonary air leak sounds in the swine model of FIGS. 51 and 52.

FIGS. 51 and 52 are a series of plots showing the correlation between airway pressure and sound pressure level in swine lung with air leak.

### DETAILED DESCRIPTION OF EXEMPLARY EMBODIMENTS

Reference will now be made to several embodiments of the present invention(s), examples of which are illustrated in the accompanying figures. Wherever practicable similar or like reference numbers may be used in the figures and may indicate similar or like functionality. The figures depict embodiments of the present invention for purposes of illustration only. One skilled in the art will readily recognize from the following description that alternative embodiments of the structures and methods illustrated herein may be employed without departing from the principles of the invention described herein.

All terms defined herein should be afforded their broadest possible interpretation, including any implied meanings as dictated by a reading of the specification as well as any words that a person having skill in the art and/or a dictionary, treatise, or similar authority would assign thereto.

The terms, “for example”, “e.g.”, “optionally”, as used herein, are intended to be used to introduce non-limiting examples. The phrases “in one embodiment” and “in some embodiments” as used herein do not necessarily refer to the same embodiment(s), though they may. Furthermore, the phrases “in another embodiment” and “in some other embodiments” as used herein do not necessarily refer to a different embodiment, although they may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.” In addition, the terms “comprises” and “comprising” when used herein specify that certain features are present in that embodiment, however, this phrase should not be interpreted to preclude the presence or addition of additional steps, operations, features, components, and/or groups thereof.

With the foregoing prefatory comments in mind, pulmonary air leak is the most common complication of lung surgery, with air leaks that persist longer than 5 days representing a major source of post-surgery morbidity. Clinical management of air leaks is challenging due to limited methods to precisely locate and assess leaks. Here, we present a sound-guided methodology that enables rapid quantitative assessment and precise localization of air leaks by analyzing the distinct sounds generated as the air escapes through defective lung tissue. Air leaks often present after lung surgery due to loss of tissue integrity at or near a staple line. Accordingly, we investigated air leak sounds from a focal pleural defect in a rat model and from a staple line failure in a clinically relevant swine model to demonstrate the high sensitivity and translational potential of this approach. We identified that: (i) pulmonary air leaks generate sounds that contain distinct harmonic series; (ii) acoustic characteristics of air leak sounds can be used to classify leak severity; and (iii) precise location of the air leak can be determined with high resolution (within 1 cm) by mapping the sound loudness level across the lung surface. Our findings suggest that sound-guided assessment and localization of pulmonary air leaks could serve as a diagnostic tool to inform air leak treatment strategies.

Risk factors for pulmonary air leak include lung surgery and biopsy, chest trauma, mechanical ventilation, and underlying lung disease, such as COVID-19. Management of pulmonary air leak is difficult, as methods to assess and locate leaks are limited, contributing to increased length of hospital stay and risk of complications. We discovered that pulmonary air leaks produce sounds with distinct acoustic signatures that may offer diagnostic and prognostic insight. Here, we report a sound analysis modality that allows accurate assessment of air leak detection/severity and treatment strategies during video-assisted thoracic surgery (VATS) or thoracotomy procedures, as well as location through acquisition and quantification of air leak sounds. This acoustic evaluation of air leaks could enable precise patient-specific treatment via guided delivery of lung sealants or other therapeutics to the exact location of the air leak.

Pulmonary air leak is a common complication of lung surgery, occurring in up to 60% of patients undergoing lung resection, frequently due to faulty staple-lines through compromised tissue. Pulmonary air leaks range in severity from ‘mild leaks’ that often resolve spontaneously, to more serious ‘prolonged leaks’ that require up to weeks to heal and often necessitate additional interventions. Prolonged air leaks that persist beyond 5 days substantially increase the risk of complications, such as empyema and pneumonia, and are associated with increased hospital length of stay and cost. Patients with severe air leaks develop pneumothorax, which can rapidly lead to tension pneumothorax, cardiovascular collapse, and death. Among the diverse population of lung surgery patients, most have underlying lung disease including chronic obstructive pulmonary disease (COPD), interstitial lung disease (ILD), and lung cancer, which predisposes them to severe, prolonged air leaks. In these patients, to quantify and localize air leaks or assess prognosis remains challenging. Methods to objectively assess severity and precisely locate pulmonary air leaks would enable targeted intervention and treatment.

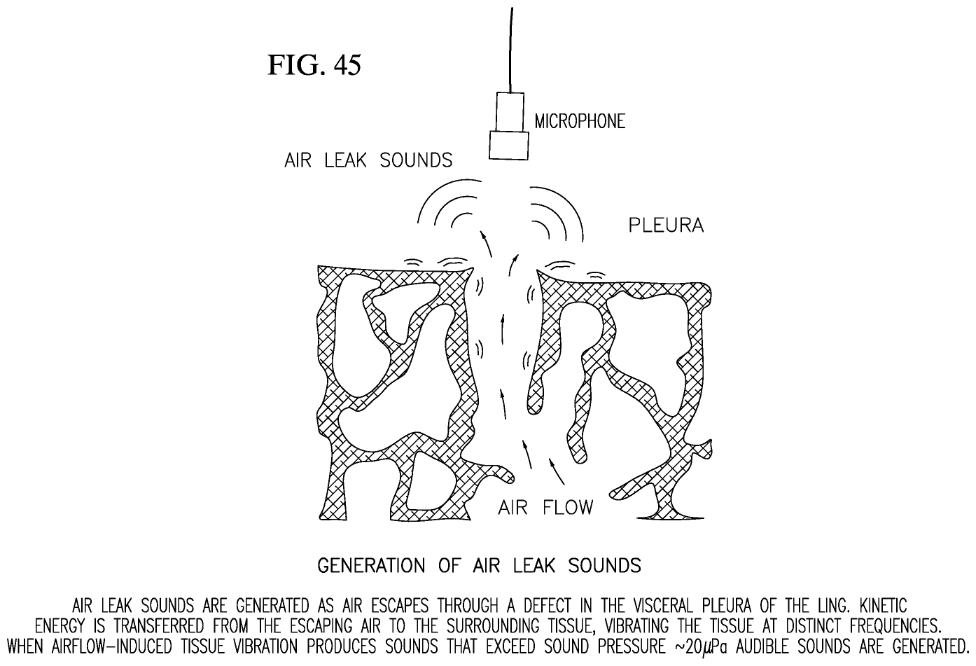

For centuries, clinicians have relied on qualitative auscultation of lung sounds generated by airflow within the respiratory tract to diagnose respiratory conditions. The properties of a periodic acoustic wave (e.g., frequency, amplitude, wavelength, velocity) determine loudness, spectral density, and pitch, which are perceived as sound. As fluid passes by a structure, energy is transferred from the fluid to the structure, causing vibration that produces sound with intensities and frequencies dependent on the amplitudes and modes of vibration. For example, the primary mechanism of human voice production is oscillation of the vocal cords produced by airflow through the glottis (fundamental frequency range: 110-300 Hz). Significantly, we discovered that airflow through defective visceral pleura, i.e., pulmonary air leak, also causes oscillations in the surrounding lung tissue, resulting in distinct, quantifiable air leak sounds (FIG. 45). This discovery led us to hypothesize that acoustic signatures of these sounds provide quantifiable information about the air leak, such as leak severity and location.

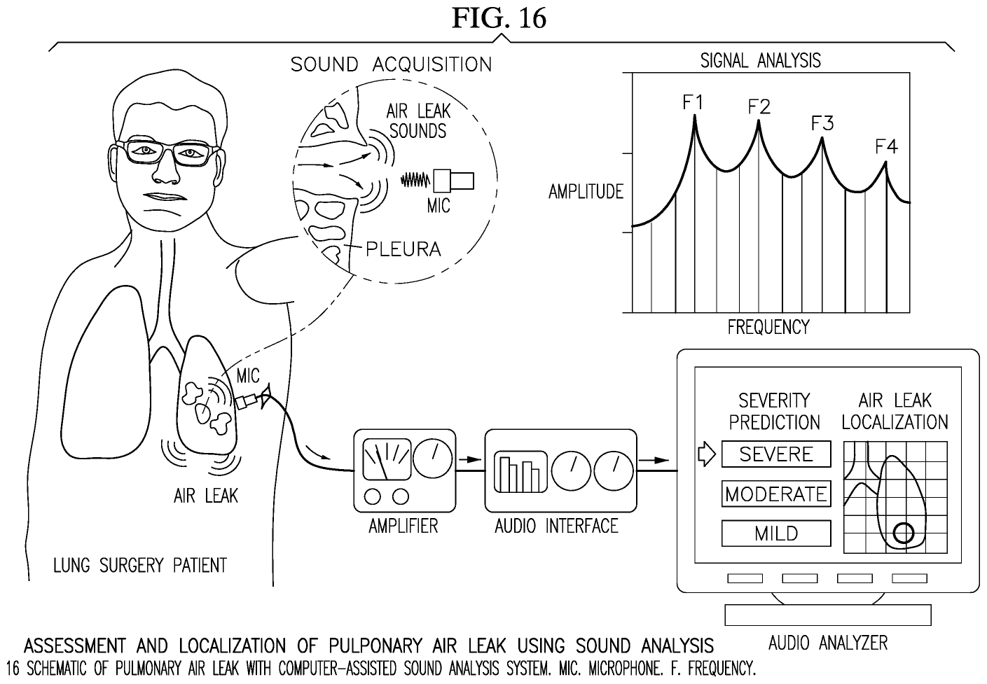

Clinical methods used to detect and assess pulmonary air leak remain limited, and do not precisely assess leak severity or locate leak site. We hypothesized that quantitative analysis of air leak sounds could enable detection, severity assessment, and precise localization of pulmonary air leak. To test this hypothesis, we developed a sound analysis system and methodology to evaluate pulmonary air leak in situ (FIGS. 16-17 and FIG. 18). In this study, we show that pulmonary air leaks can be evaluated by analyzing air leak sound signals. Further, we demonstrate that air leak sound profiles can be used to classify severity and localize the air leak with significantly higher resolution than any currently available methods. Using a clinically relevant large animal model (swine) of free-flowing air leak in an open thoracotomy, we evaluated post-surgical air leaks due to staple-line failure, and applied our quantitative sound-guided methodology to distinguish mild and severe air leaks and to precisely localize air leak sites. We identified that air leak sounds contain frequencies that comprise harmonic series, a finding not previously reported. Altogether, our results indicate that pulmonary air leak sounds are quantifiable in a clinically relevant setting, and could be used for real-time severity assessment and precise localization of air leaks.

The disclosed subject matter generally relates to methods, device and integrated systems for detecting and repairing a pulmonary air-leak in a patient.

In one aspect, a device, such as a vest worn by a patient, has an array of embedded microphone sensors arranged in a specific geometric orientation on the anterior and/or posterior sides of vest. The sensors collect acoustic signals produced by the air leak. An acoustic analyzer uses filtering noise algorithms for noise cancellation and isolation of the air leak signals. Location of the air leak is determined by sound localization algorithms assessing different times of arrival and intensity of the air leak soundstem in conjunction an air-leak locator and a therapeutic delivery system and methods of using the system to treat a patient with pulmonary air-leak.

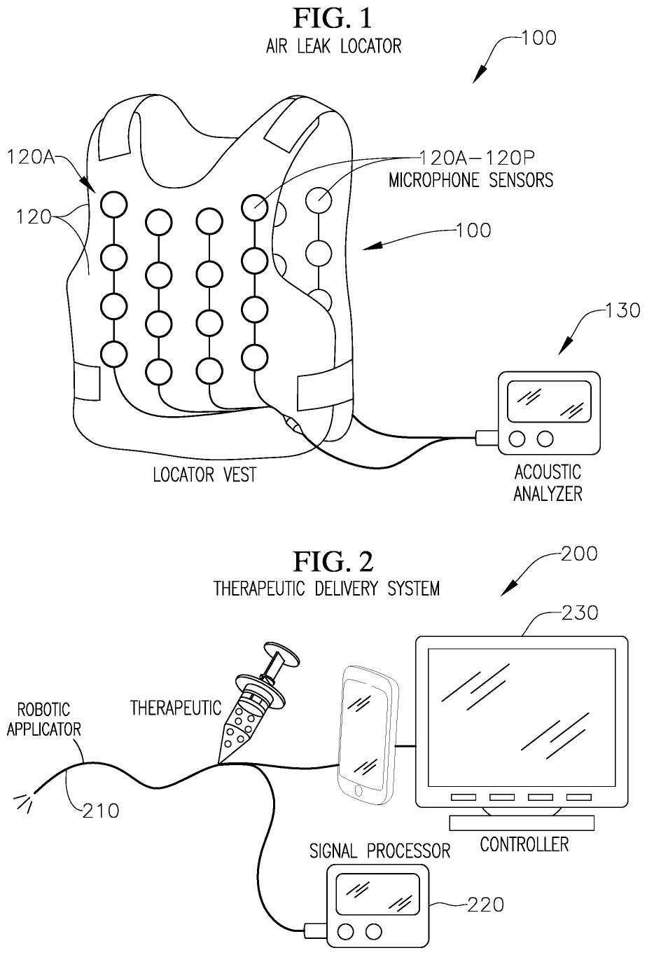

In one aspect, referring to FIGS. 1-2, a system is provided for the non-invasive detection and location of an air leak. The system includes an air leak locator device 100 that detects and locates pulmonary air leak, and a therapeutic delivery system 200 that navigates a robotic applicator 210 to the site of the air leak and delivers a therapeutic payload to treat the leak.

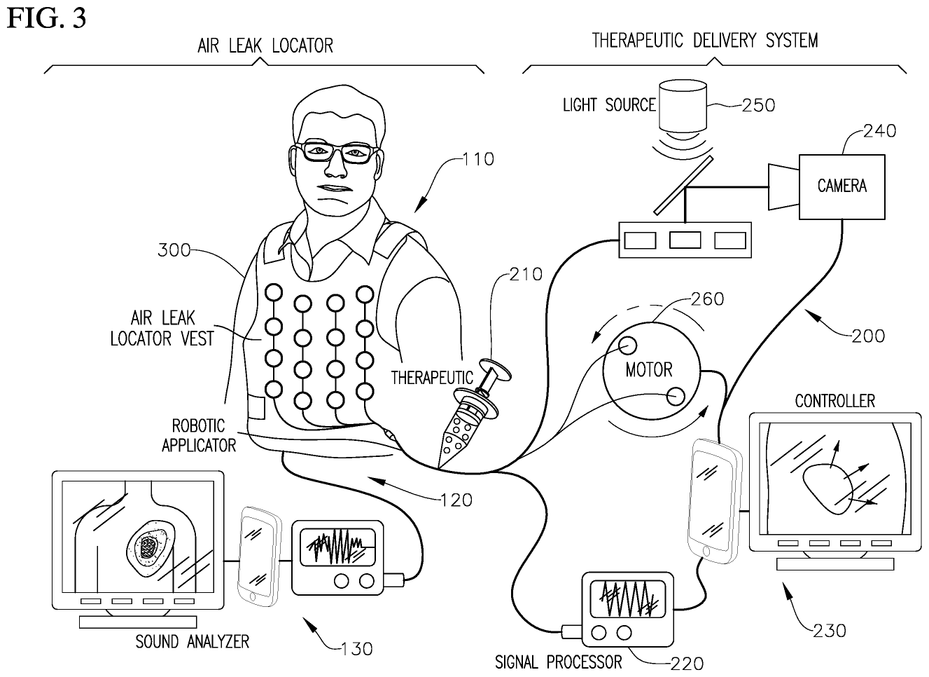

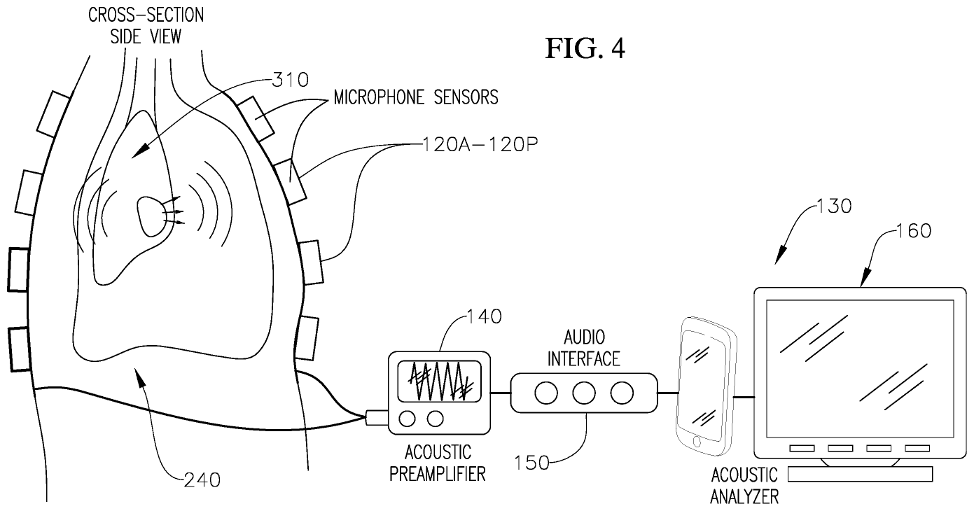

In one embodiment, referring to FIG. 3, the air leak locator device 100 comprises a vest 110 having an array of microphone sensors 120 that collects unique acoustic signals produced by the air leak. The multiple microphone sensors 120a to 120p are arranged in a specific geometric configuration (e.g., 6×6) and are integrated into the vest 110 worn by a patient 300 with pulmonary air leak 310 as shown in FIG. 3. The vest 110 ensures microphone sensors 120a-120p are in close contact with the upper and mid trunk of the patient 300 to noninvasively collect acoustic signals (i.e., sounds) generated in the chest of the patient. Acoustic signals detected by the microphone sensor array 120 are amplified by an acoustic amplifier device 140 and conveyed via an audio interface device 150 to a computerized acoustic analyzer 130 that identifies the location of the air leak 310 in the chest. A display 160 may show the location of the leak in the patient. The microphone can be in a sensor array in a 3×3 configuration for detecting breathing sounds of a human subject.

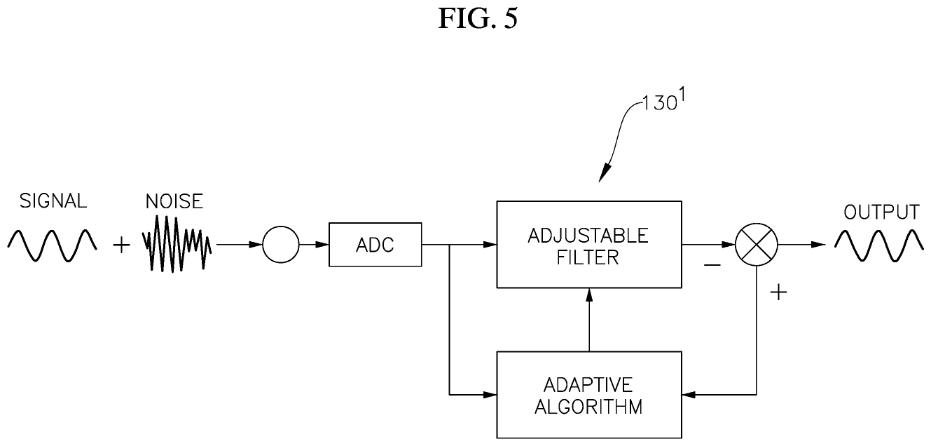

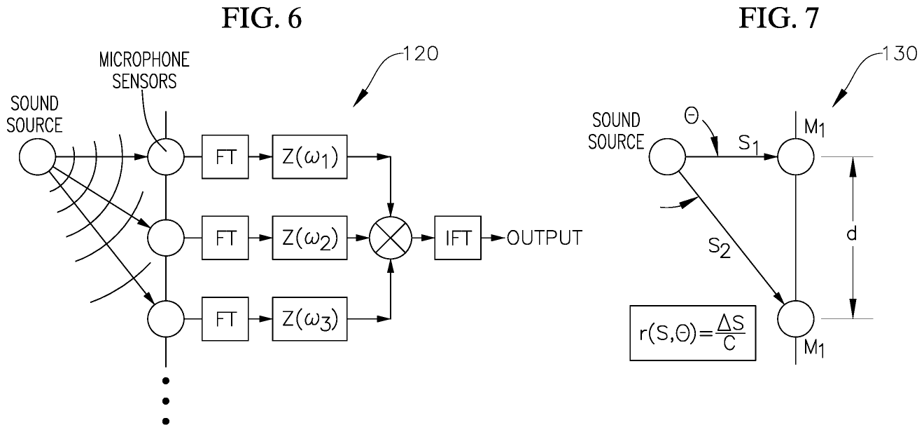

Referring to FIG. 5, in one embodiment, the acoustic analyzer 130′ uses noise filtering algorithms (e.g., adaptive noise cancellation) to isolate the unique sound (signal) produced by the air leak 310 and to filter out noise (e.g., heartbeat, breathing, ambient sounds). Referring to FIGS. 6 and 7, location of the air leak is determined by a programmed computer 130 using sound localization algorithms that assess differences in time-arrival and intensity of the air leak sound measured by individual microphone sensors embedded in the locator vest. The programming takes into account that due to the different distances between individual microphone sensors and the source of the sound (air leak), sounds produced by the air leak arrive at each microphone sensor at different times (time-arrival difference). Furthermore, due to the inversely proportional relationship between sound intensity and distance to sound source, sound intensities measured by individual sensors also differ (intensity difference). The acoustic signals collected by individual microphone sensors are filtered and processed using customized MAT LAB programs to analyze the unique sounds produced by air leak. Normal breathing sounds recorded in a healthy human subject with normal pleura show characteristic sound profiles with relatively increased intensity during inspiration and initial expiration, and relatively decreased intensity at the end of expiration.

The spectral density of the normal signal reveals that a normal breath sound mainly contains acoustic waves in the frequency range of 150-450 Hz. In contrast, breathing sounds recorded in a human subject with inflamed pleura (pleuritis), such as that caused by air leak, show a distinct, abnormal sound profile with radically reduced sound intensity. The spectral density of the abnormal signal reveals that an abnormal breath sound contains acoustic waves in the frequencies of 200 Hz and 450 Hz. These measurable differences in air leak sounds can be used to detect and determine the location of air leaks.

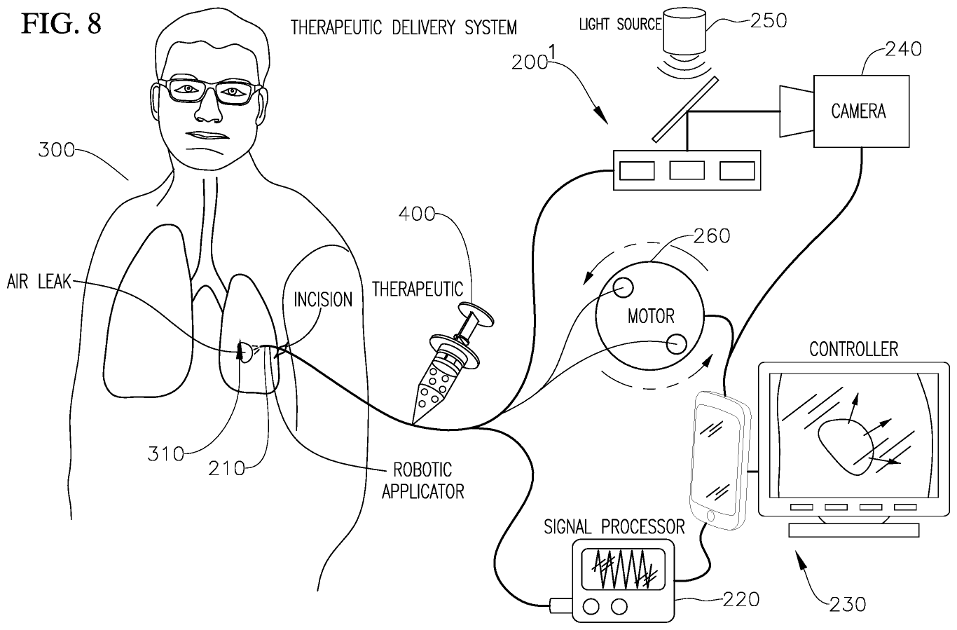

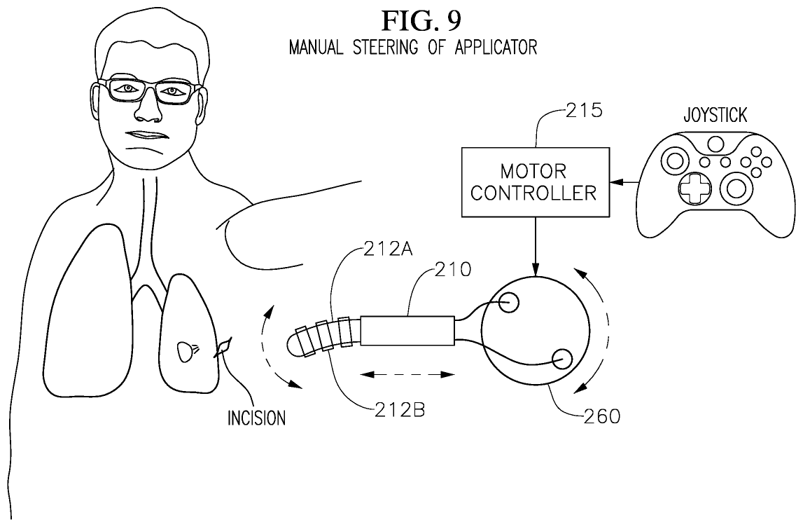

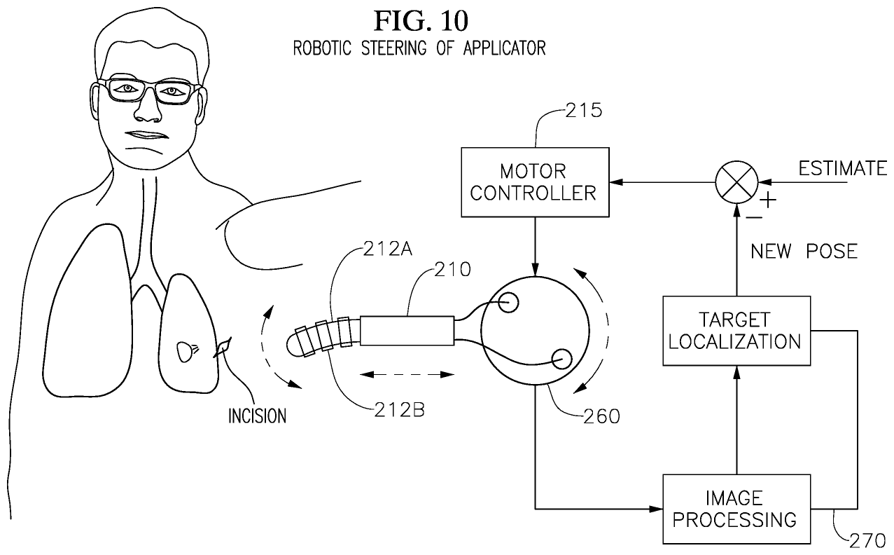

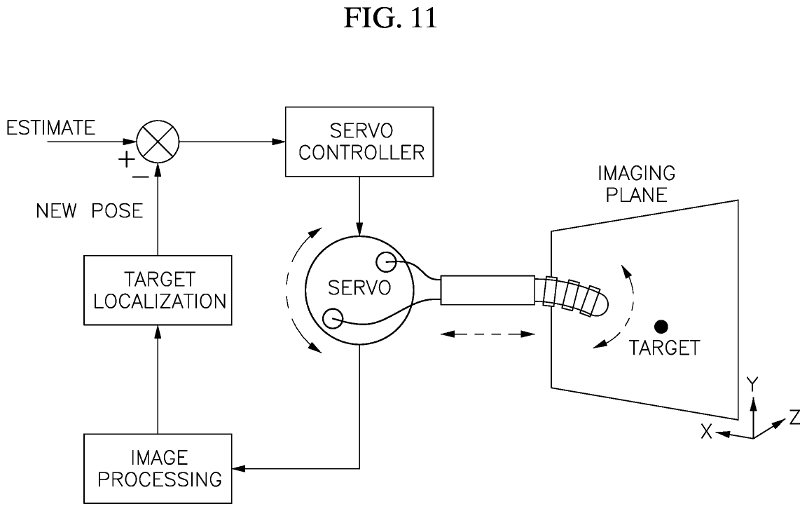

In one embodiment (see FIG. 3), the therapeutic delivery system 200 comprises a light source 250, camera 240, signal processor 220, servo motor 260, controller 230, and robotic applicator 210. The system 200 is configured to deploy the robotic applicator 210 to deliver a therapeutic 400 (e.g., sealant) locally and directly to the site of air leak 310 to treat the leak, as shown in FIG. 8. Referring to FIG. 9, steering (deflection and translational movements) of the applicator 210 is performed by controlling the rotational angles of one or more motors 260 attached to the device by pulling wires 212a and 212b. The one or more motors 260 are directed by a controller 215, such as a joystick, that is operated manually by a health care provider, or fully or partially automated by computer vision guidance 270, as shown in FIG. 10, including visual servoing, where the optical information obtained by the optical fiber imaging probe is processed and fed back to individual motors that steer the applicator. By utilizing computer vision guidance 270, the speed and accuracy of therapeutic delivery to the targeted site can be significantly improved. With computer vision-assisted guidance 270, the prototype device 210′ demonstrated continuous locating and tracking of a target, as shown in FIG. 11. The motorized steerable applicator (length: 60 cm, outer diameter: 5 mm) features a flexible polymeric conduit (outer diameter: 3 mm) with guide-wire disks (outer diameter: 5 mm) attached to the outer surface of the distal end of the tube. Four pulling wires (diameter: 300 μm) adhere to the distal tip at 90° relative to each other around the tubular body, passing through small holes (diameter: 350 μm) in the guide-wire disks, and connecting to computer-controlled motors.

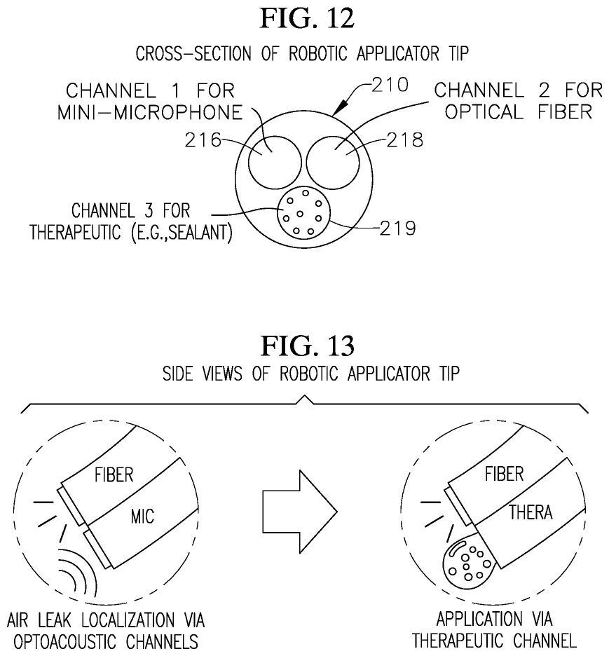

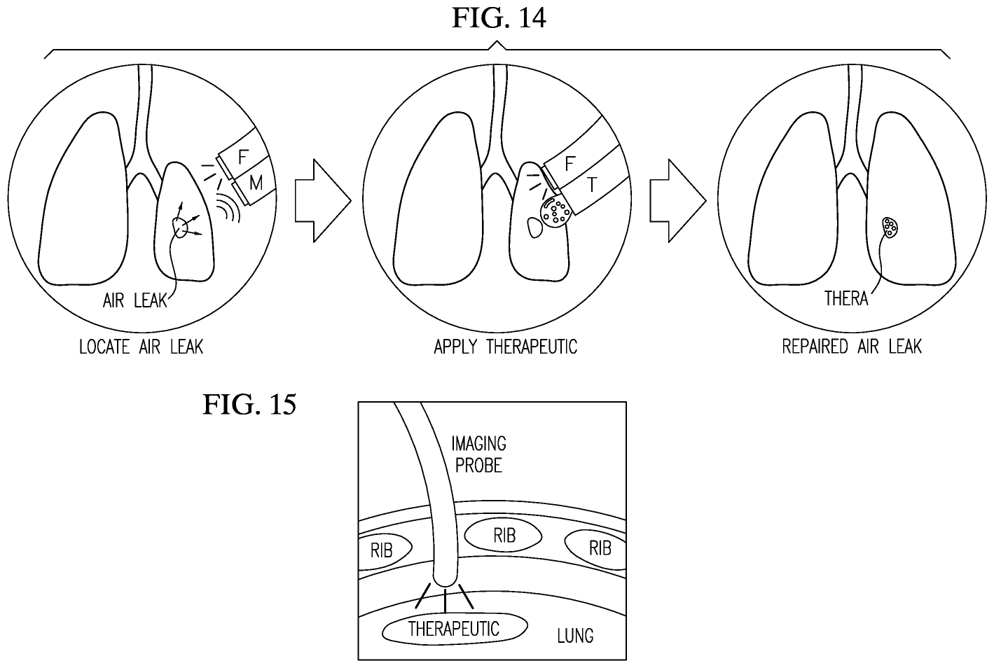

In one embodiment, the applicator 210 has a longitudinal body including first 216, second 218 and third 219 channels along at least a portion of its length configured to receive a microphone, optical fiber, and therapeutic deliverable, respectively, as depicted in FIG. 12. An integrated mini-microphone (diameter: 1 mm) continuously collects unique air leak sounds that guide the robotic applicator to the precise source of the sound (air leak site) prior to delivery of the therapeutic payload to the site of the air leak. Navigation is supported by an integrated flexible optical microfiber imaging probe (diameter: 300 μm) coupled to a custom microscope (light source, camera). The applicator 210 also has a conduit (diameter: 1-2 mm) for therapeutic delivery 219. Referring to FIG. 13, after locating the air leak, a therapeutic is topically applied through the therapeutic conduit 219. Thus, following applicator 210 navigation to the air leak site in the chest 310, the therapeutic payload 400 is deployed via the therapeutic channel 219 to treat and repair the air leak 310, as shown in FIG. 14.

Referring to FIG. 15, the system was used to treat lungs of a rat by inserting the optical fiber imaging probe in the chest of the rat and delivering a therapeutic payload. Using real-time image processing, the rat lung pleura and delivered therapeutic payload were visualized during and after delivery. Examples of therapeutic deliverables include but are not limited to: cells (e.g., mesenchymal stem cells), exosomes, drugs (e.g., small molecules, biologics, and chemotherapeutics), biomaterials, radiation (e.g., heat, light). Applications for the use of this system extend beyond air leak, and include but are not limited to navigation and therapeutic delivery to: lung parenchyma, lung pleura, chest wall, and other intrathoracic, intra-abdominal, intravaginal, etc. surfaces.

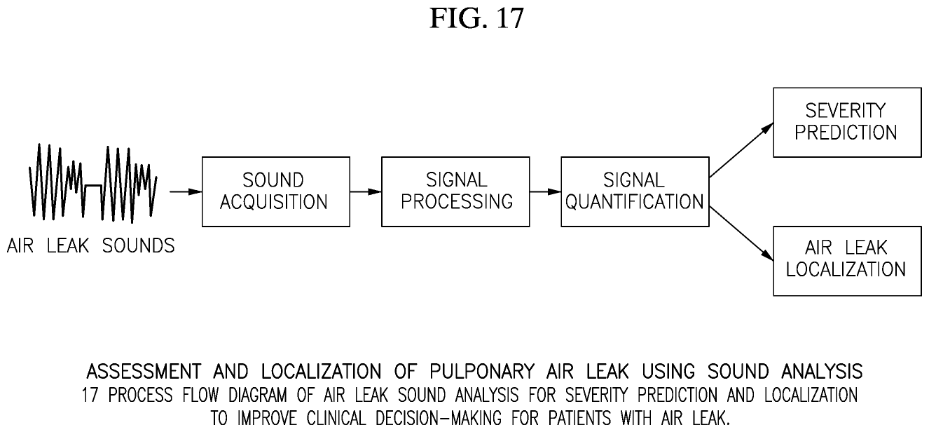

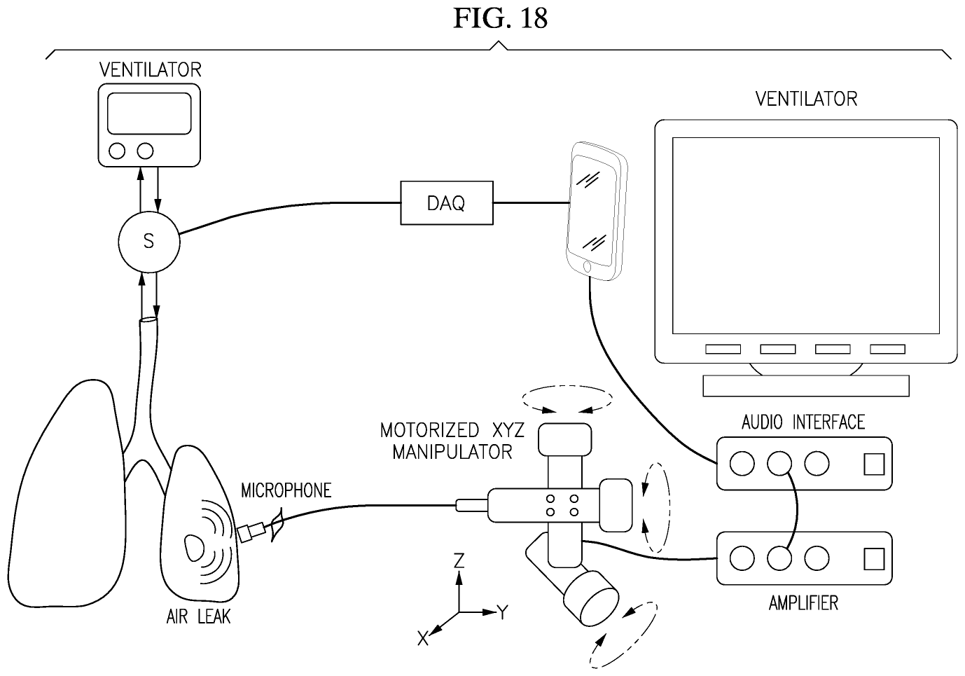

Quantitative sound analysis of pulmonary air leak. Clinical methods used to detect and assess pulmonary air leak remain limited, and do not precisely assess leak severity or locate leak site. We hypothesized that quantitative analysis of air leak sounds could enable detection, severity assessment, and precise localization of pulmonary air leak. To test this hypothesis, we developed a sound analysis system and methodology to evaluate pulmonary air leak in situ. The sound acquisition and analysis system comprised a miniature microphone (diameter: 5.6 mm; length: 3 m), motorized stage to position the microphone, signal amplifier, audio interface, and custom-written MATLAB codes (FIGS. 16 and 18). Notably, a standard thoracoscopic port has an inner diameter of 12 mm, thus allowing easy insertion of the mini-microphone for minimally invasive sound analysis in situ. Sound signals obtained with the microphone passed through the amplifier and audio interface to the computer for sound processing and quantification using custom-written MATLAB scripts, which removed ambient noise, amplified signals, and quantified acquired sounds to predict severity and determine air leak location (FIG. 17). To validate this methodology, we applied quantitative sound analysis in rat and swine models of pulmonary air leak and determined specific acoustic signatures associated with air leaks under different injury states and ventilatory conditions.

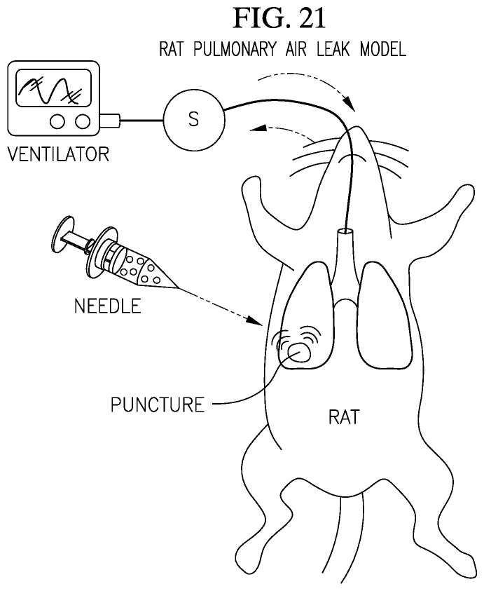

Rat model of pulmonary air leak. Post-surgical air leak is caused by a perforation in the visceral pleura that often occurs along or near a staple line due to failure of the staples or tearing of lung tissue. Defects can occur (i) focally at a single staple or (ii) along a series of consecutive staples, and range in size from <1 mm to >1 cm. In this study, we investigated small defects resulting from focal puncture (i.e., <1 mm) in a rat model of pulmonary air leak to assess the sensitivity of our system to detect minor leaks that result from failure of a single staple (FIGS. 21-30). Rat lungs provide a suitable model to test sensitivity of our methodology and system because air pressures and tidal volumes of rat lungs are significantly lower than those of human lungs, corresponding to lower pressure drop (AP) across the lung alveoli and thoracic space, resulting in quieter air leak sounds. By validating efficacy of the sound analysis in rat lungs, we demonstrated high sensitivity and accuracy of our system and methodology.

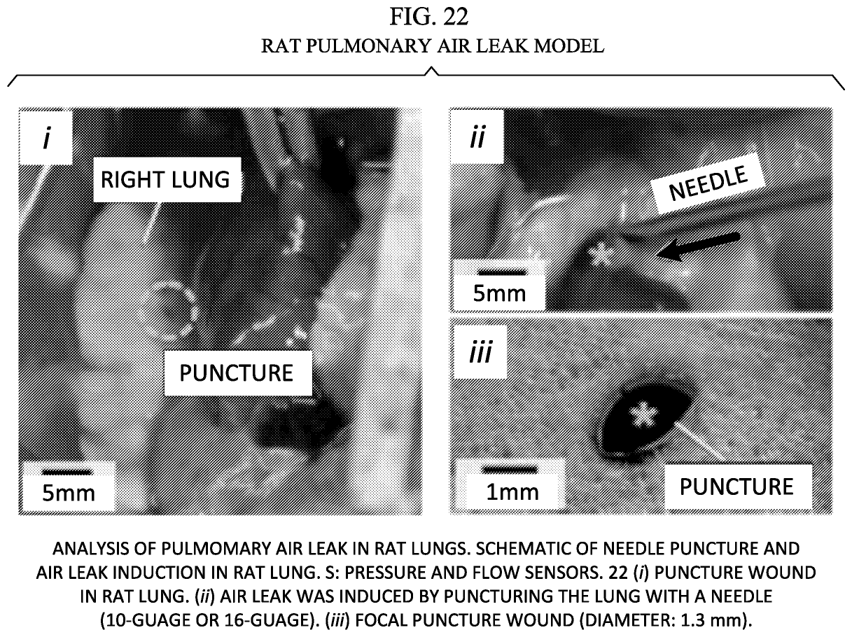

To induce air leak in rat lung, we punctured the visceral pleura of intubated rats that were ventilated with a tidal volume of 6 mL/kg at a ventilation frequency of 70 breaths per minute (bpm) using an 18-gauge (diameter: 1.27 mm) or 16-gauge (diameter: 1.65 mm) needle (FIG. 22). Airway pressures were continuously monitored using a small animal ventilator, pressure sensors, and custom-written MATLAB program (FIG. 21). Air leaks were confirmed by observation of decreased air pressure and compliance, as indicated by flattened peaks of the pressure curves. Focal puncture wound induced with 18-gauge and 16-gauge needle led to declines in peak inspiratory pressure of 39% (from 16.2 to 9.9 cm H₂O; and 48%, respectively (from 16.2 to 6.7 cmH₂O).

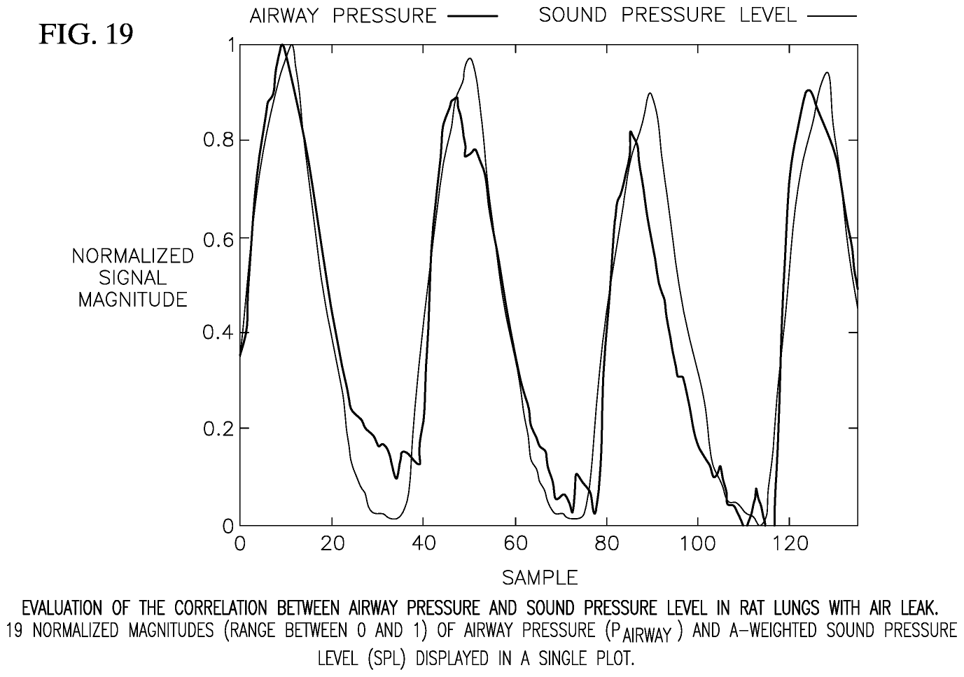

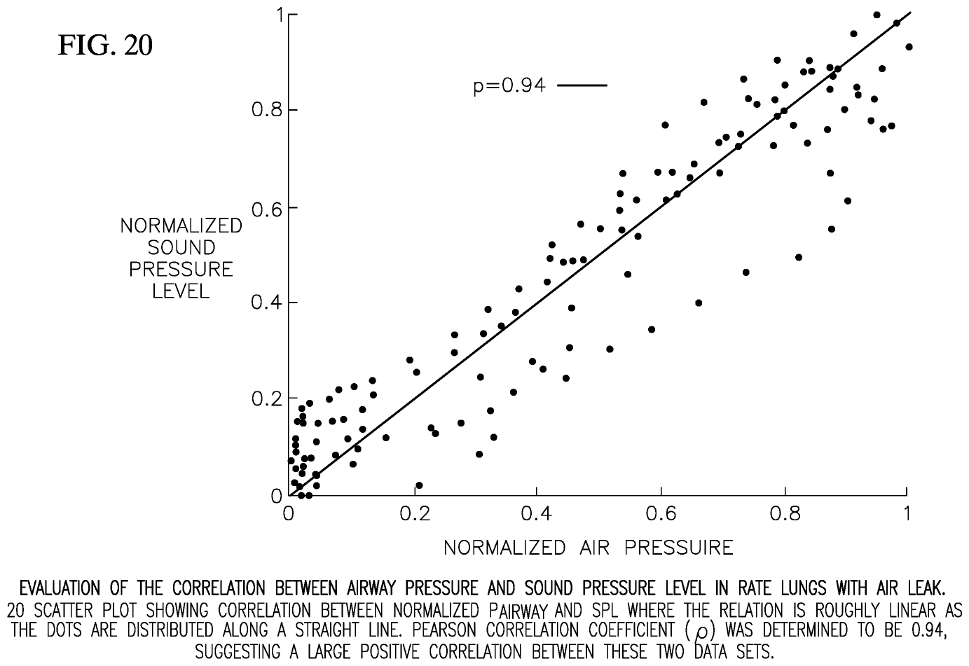

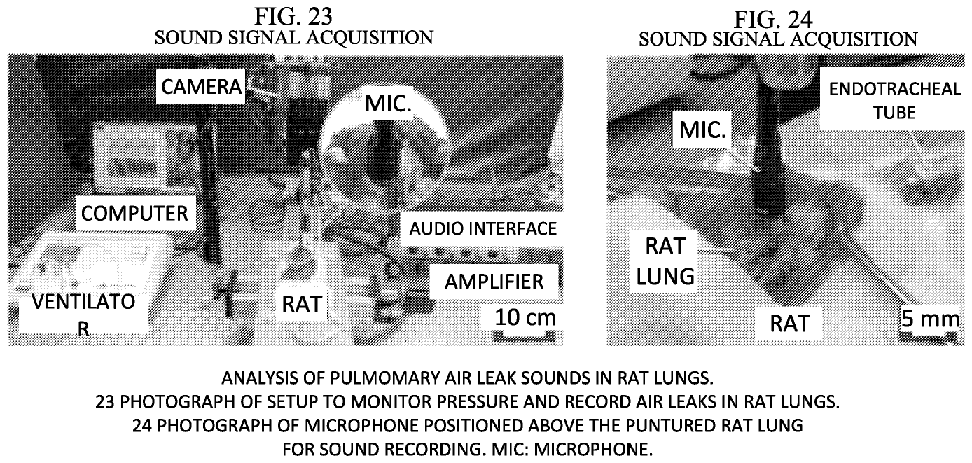

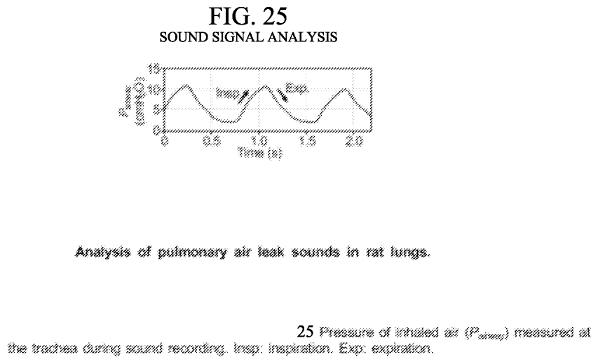

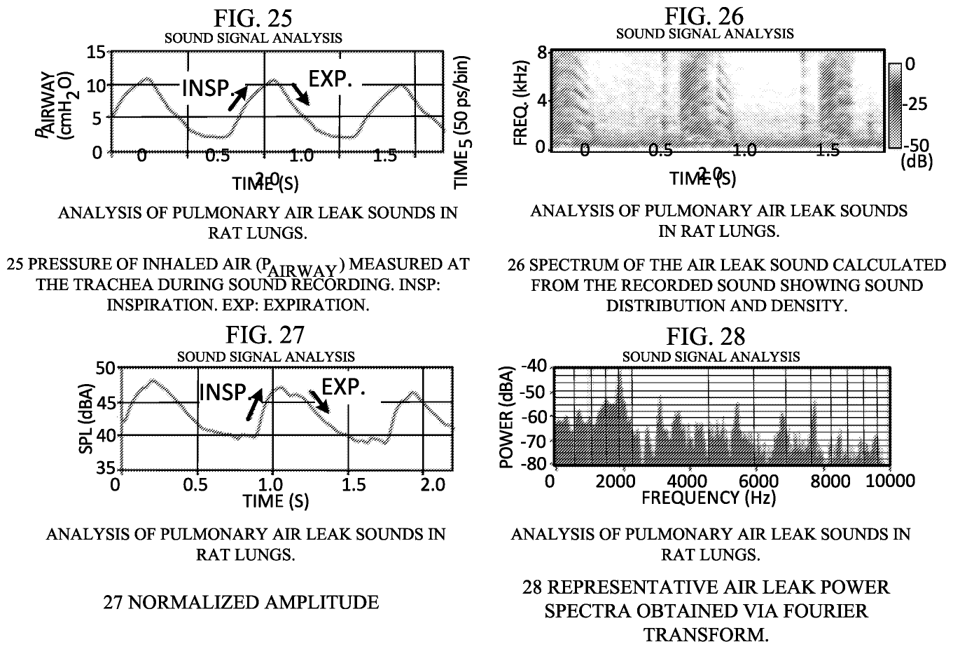

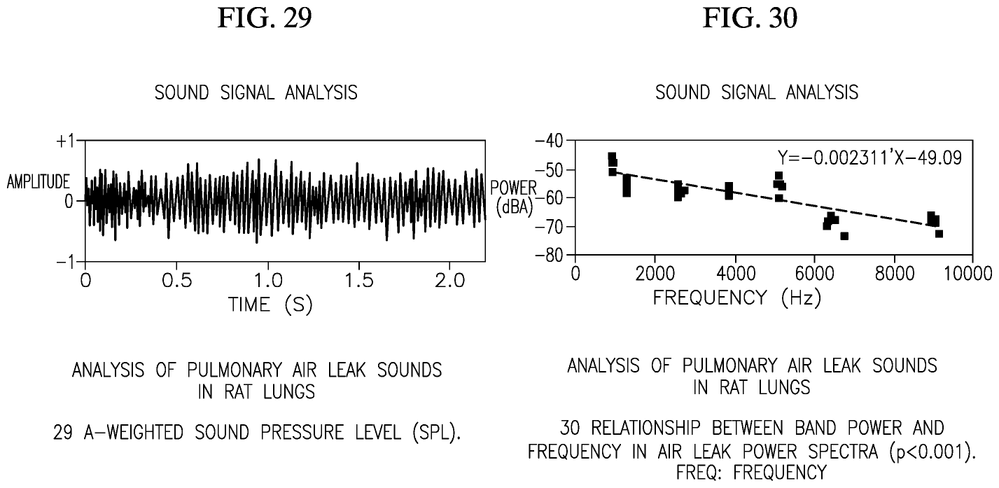

Detection of pulmonary air leak in rat using acoustic evaluation of air leak sounds. We investigated whether changes in peak inspiratory pressure resulting from air leak would be accompanied by corresponding changes in sound frequency at the air leak site. Air leak sounds were recorded and analyzed across three respiratory cycles using our custom-built sound system (FIGS. 23-24) and all measurements were repeated three times. Because airway pressure (P_{airway}: 2.1-10.7 cmH₂O) (FIG. 25) was always greater than ambient pressure (P_{Amb}: 0 cmH₂O), we presumed that during positive pressure ventilation no air flowed back into the lung through the leak site, and that acquired sounds represented the acoustic signal resulting from air escaping through the punctured pleura (FIG. 29). Loudness was calculated as sound pressure level (SPL), which was quantified in A-weighted decibel (dBA), and plotted against time to determine if intensity and periodicity of the sound correlated with Pairway (FIG. 27). Analysis showed a positive association between Pairway and SPL as the shapes of these two curves closely resembled each other (FIGS. 25 and 27). We quantified this correlation and determined a positive linear relationship between sound pressure level and air pressure with Pearson correlation coefficient (ρ) of 0.94 (FIGS. 19, 20, 25 and 29). Pressure difference (ΔP) between inside (Pairway) and outside (P_{Amb}) the lung is the major driving force that causes air leak. Due to difficulties in accurate measurement of Palveolar, we instead measured Pairway. While the two values are related, they are not the same. During normal ventilation, there is a pressure drop across the airways, dependent on driving frequency and airway structure. Accordingly, the loudest (47.5 dBA) and quietest (39.8 dBA) acquired sounds corresponded to the maximum (10.7 cmH₂O) and minimum airway pressure (2.1 cmH₂O), respectively, in each respiratory cycle. Loudness of the air leak sounds increased more rapidly during inspiration (24.84 dBA/s) than expiration (14.53 dBA/s), suggesting that air leak loudness depends on ventilation parameters (i.e., Pairway, tidal volume, respiratory rate) which contribute to the total volume of air loss through the pleural defect.

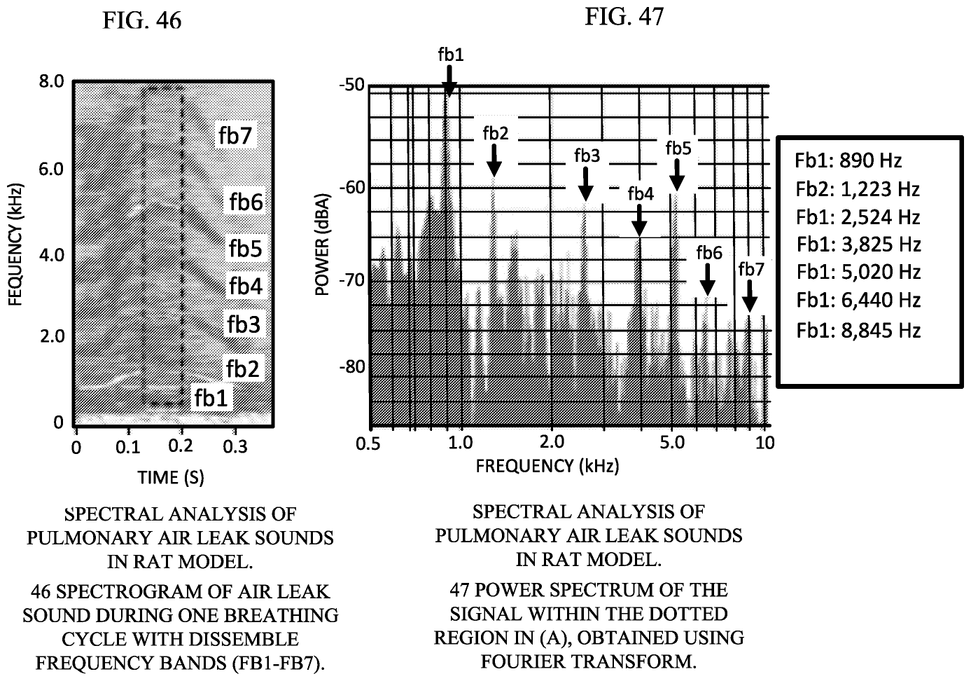

To quantify air leak sound signatures, we analyzed the frequency distribution of the air leak sounds acquired from the rat model of air leak through spectral analysis (FIG. 26). Sound spectrograms were generated to assess time-varying intensity and frequency throughout the respiratory cycle. The spectrograms revealed that the air leak sounds contained a group of sound bands, each with a distinct frequency that increased and then decreased (FIGS. 46-47) in patterns similar to those of P_{airway}, amplitude, and sound loudness (FIGS. 25, 27, 29). This suggests that both loudness and frequency of air leak sounds can be used to characterize and quantify air leak. We then extracted the air leak sound near the plateau of the curves in the spectrogram (dotted region in FIG. 46) and obtained power spectra to compare relative intensity of the frequency bands (FIG. FIGS. 28, 47). We plotted the regions of air leak sound spectrograms that contained frequency bands, which consistently corresponded to inspiratory peaks. Seven distinct frequency bands (fb1-fb7) with amplitude above-80 dB were identified between 0 and 10 kHz, each with narrow and concentrated frequencies. The lowest frequency band (i.e., fb1: 890 Hz) had maximum intensity among the seven bands, and was determined to be noise generated by the ventilator. While intensity of the sound bands generally decreased with frequency, the band that centered at 5,020 Hz (fb5) had the second strongest intensity. A harmonic sound consists of a series of sound frequencies at integer multiples of the fundamental frequency. The ratios of fb3-7 (frequency range: 2,524-8,845 Hz) to fb2 (frequency: 1,223 Hz) were approximately 2, 3, 4, 5, and 7, respectively, indicating these sound bands are a harmonic series. To determine the relationship between band frequency and sound intensity, we performed a linear regression on the identified frequency bands generated by air leak in rat, and found a statistically significant inverse correlation (FIGS. 30, Y=−0.00231X−49.09, where X is frequency and Y is frequency band intensity, R2=0.702, p<0.001).

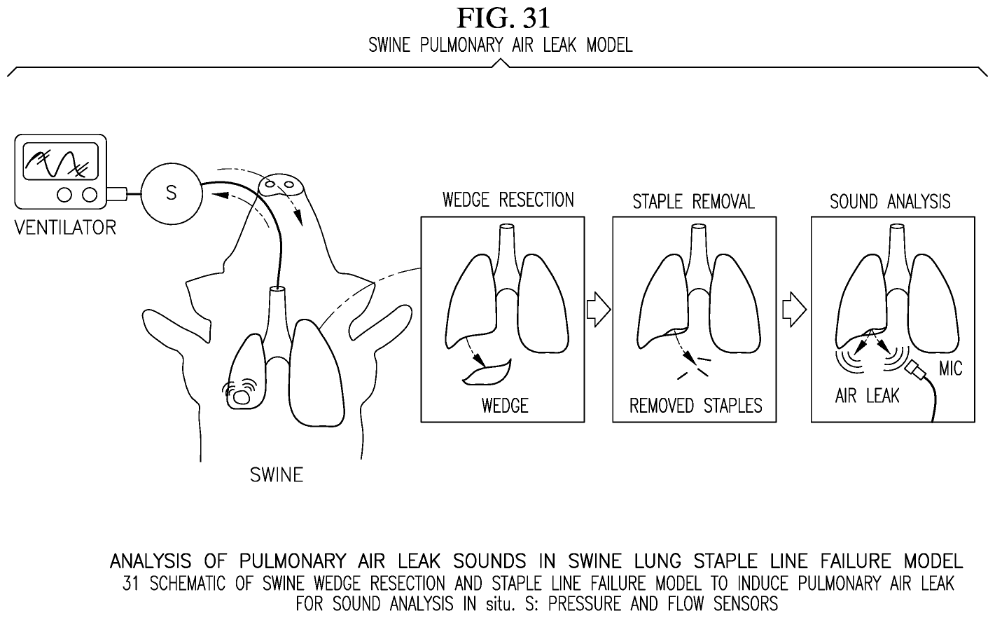

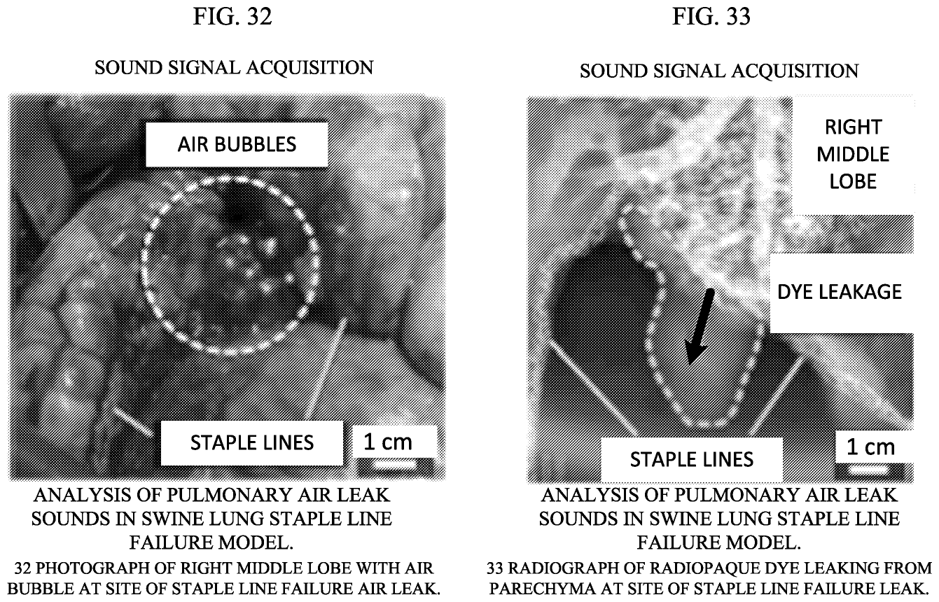

Swine model of pulmonary air leak. Swine are often used as a model for lung injury because the anatomy, size, and respiratory parameters are similar to those of human lungs. We assessed if similar air leak sounds occur in human-sized lungs and determined that the harmonic characteristic is a distinct, quantifiable feature of air leak sounds using a clinically relevant swine model of pulmonary air leak, which: (i) mimics airway pressures and volumes observed in human lungs, (ii) recapitulates post-surgical air leak due to staple line failure, and (iii) occurs in situ (FIG. 31). Wedge resection of the right middle lobe was performed using a surgical stapler. By removing up to 30% of the staples, we induced staple-line failure air leak that resulted in substantial decrease in average tidal volumes which was measured using the ventilator (i.e., 45.2 mL/breath or 27.4% loss; p<0.003. Swine lungs were ventilated with a positive end expiratory pressure (PEEP) of 5 cm H2O for all studies. Air leak was confirmed through direct observation of air bubbles at the air leak site (FIG. 32) and by radiographic visualization of radiopaque dye leakage following bronchoscopic delivery (FIG. 33).

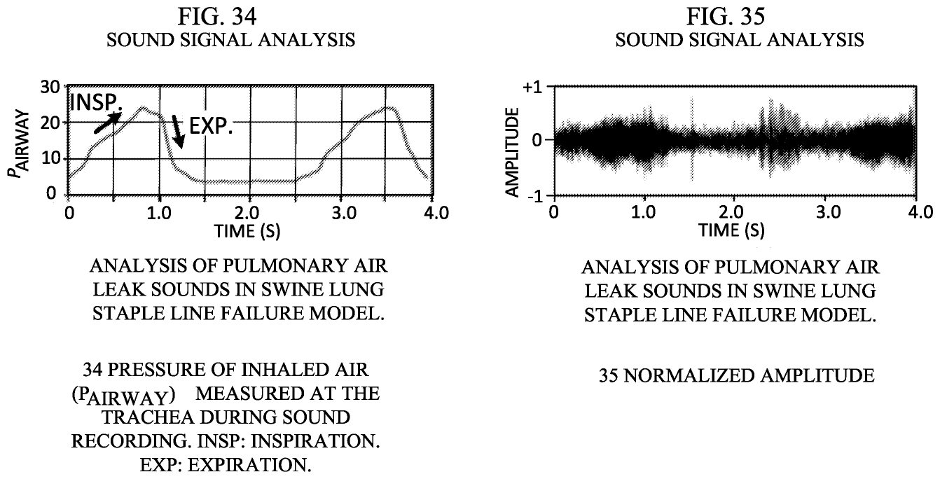

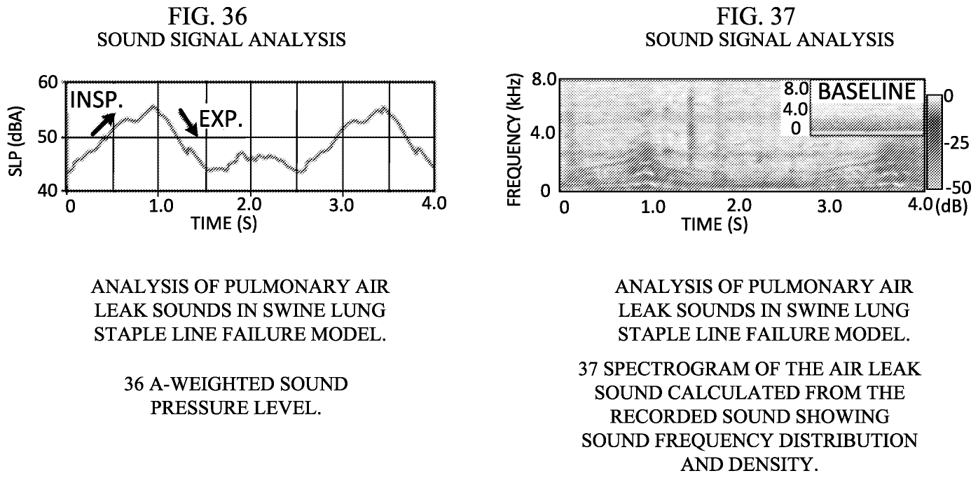

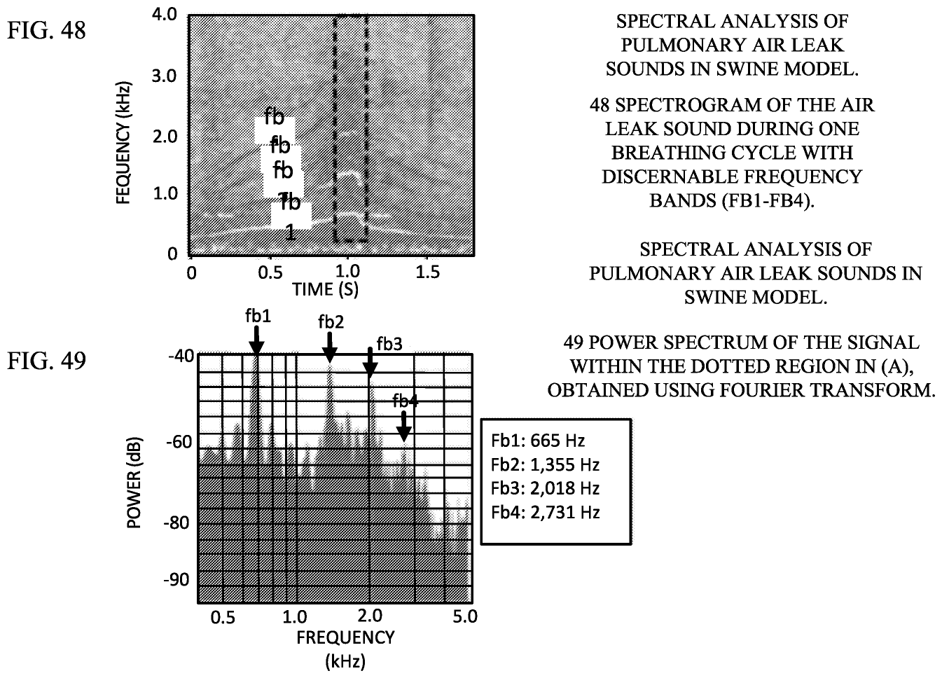

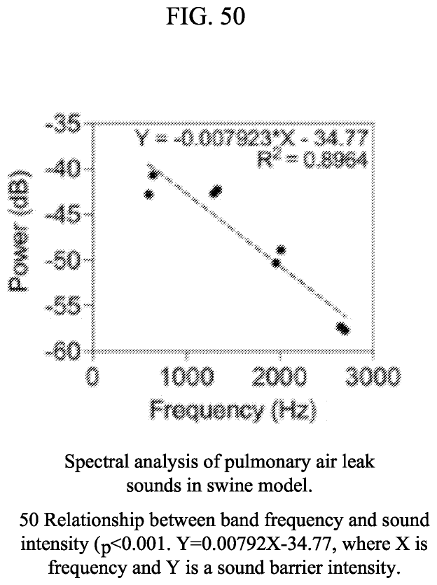

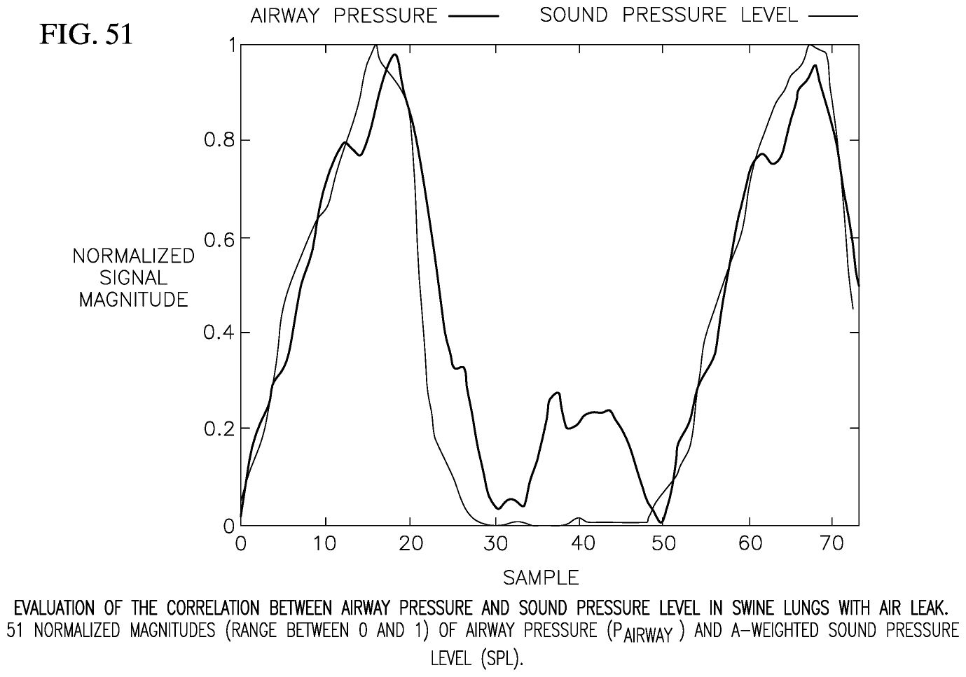

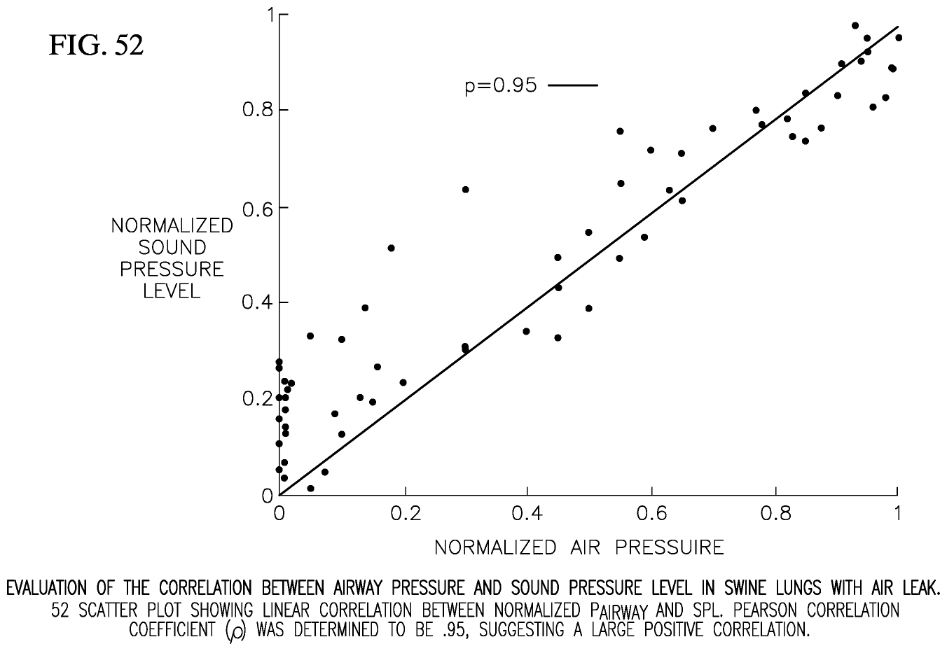

Detection of pulmonary air leak in swine using acoustic evaluation of air leak sounds. We then assessed air leak sounds in a clinically relevant setting by applying our methodology in human-sized (i.e., swine) lungs in situ. Following staple line failure, we obtained intensity, loudness, and spectrograms of air leak sounds using our sound analysis system. Swine lungs were ventilated at 15 bpm while airway pressure and tidal volume were continuously monitored (FIG. 34), and readouts obtained from at least two breathing cycles were extracted and plotted with respect to time. Consistent with observations in rat lungs, amplitude (FIG. 35), calculated loudness level (FIG. 36), and spectrogram (FIG. 37) of the air leak sounds closely correlated with ventilation parameters, as sound intensity (loudness) corresponded with Pairway during ventilation (FIG. 34). We again determined a positive linear relationship between sound pressure level and airway pressure with Pearson correlation coefficient (ρ) of 0.95 (FIGS. 51, 52). Loudness of air leak sounds in swine lungs (range: 43.4-55.5 dBA) was greater than that of rat lungs (range: 39.8-47.5 dBA), possibly due to greater AP (ΔPswine=20.2 cmH₂O; ΔPrat=18.6 cmH₂O). Swine lung spectrograms showed that air leak sound signature contained approximately four frequency bands between 0 and 5 kHz that fluctuated with Pairway (FIG. 37). While the frequency bands extended beyond 8 kHz in rat lungs (FIG. 26) and FIGS. 46-47), the bands predominantly remained below 4 kHz in swine lungs, possibly due to difference in defect size (20 mm in swine vs 1.3 mm in rat), as oscillation frequency by airflow is inversely proportional to defect length. Frequency bands with amplitude above −60 dB identified at the plateau (i.e., fb1-4) were extracted and plotted in a power spectrum (FIG. 48), in which sound intensity generally decreased with frequency, as in rat air leaks (FIGS. 46, 47). The fundamental frequency band (fb1: 665 Hz) generated the highest intensity sound. The ratios of fb2-4 (frequency range: 1,355-2,731 Hz) to fb1 were approximately 2, 3, and 4, respectively, comprising a harmonic series (FIG. 49)). We performed a linear regression on the identified frequency bands generated by air leak in swine, and found a statistically significant inverse correlation between band frequency and sound intensity (FIGS. 50, Y=−0.00792X−34.77, where X is frequency and Y is sound band intensity, R2=0.896, p<0.001 (FIG. 50).

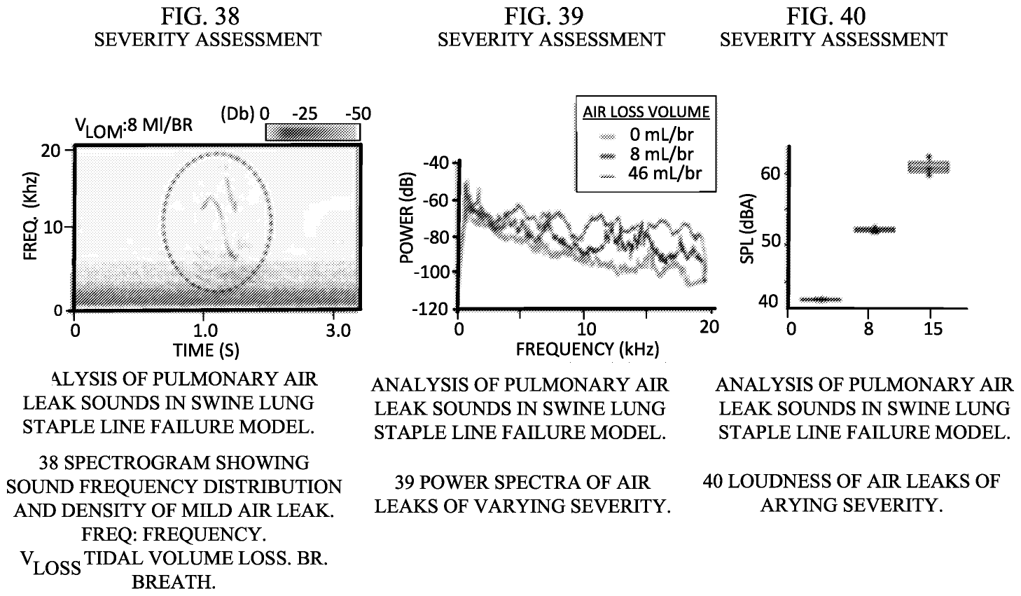

Assessment of air leak severity in swine lung using acoustic analysis. Air leak severity is typically described by the volume of air escaping from the leak site. To assess air leak severity using our sound-guided method, we modulated volume loss and analyzed the effects on air leak sounds. For a mild leak (air volume loss of 8 mL/breath), we detected multiple frequency bands of reduced sound intensities compared to those detected in severe air leak within the acquired sounds (FIGS. 38 and 39). No frequency bands were detectable on spectral analysis in the absence of air leak as normal breath sounds display energy in broad bands depending on air flow, chest wall thickness, and location of sound acquisition. In both mild (volume loss: 8 mL/breath) and moderate (volume loss: 46 mL/breath) air leaks, discernible frequency bands were observed on spectral analysis. Sounds generated due to bursting of air bubbles near air leak site were visualized as multiple vertical lines in the spectrogram. Further, we assessed the sensitivity of our sound-guided method in distinguishing subtle differences of air volume loss. Results showed that our method can accurately differentiate two different levels of air volume loss representative of mild air leak by comparison of sound intensity (52.3 dBA for 8 mL/breath and 62.1 dBA for 15 mL/breath; FIG. 40). Collectively, the results suggest that sound-guided detection and quantification of pulmonary air leak can enable rapid and accurate assessment and differentiation of air leak severity with a high degree of sensitivity.

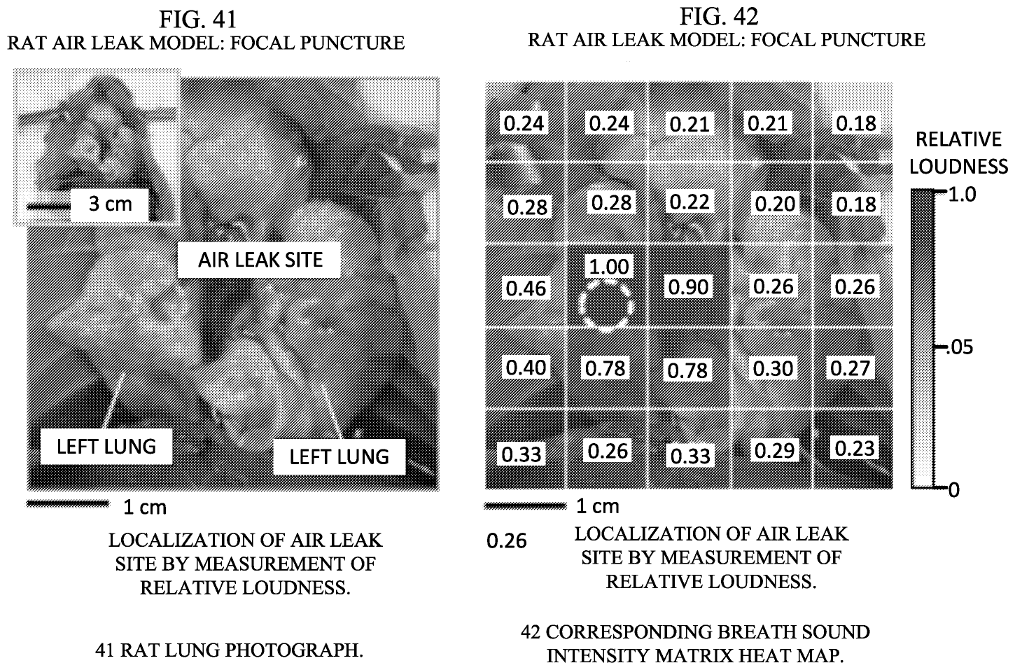

Precise localization of pulmonary air leak via sound intensity measurements. Clinical methods, such as X-ray and ultrasound, detect presence of pneumothorax resulting from air leak, and can only determine if the leak is in the left or right lung. Precise identification of the exact air leak site could facilitate targeted intervention and thus improve patient outcomes. Therefore, we investigated whether quantitative sound analysis could enable high-resolution localization of air leak site (FIGS. 41-44). Rat lungs with focal puncture air leak were continuously ventilated while a breathing sound intensity matrix was constructed. To generate breathing sound intensity matrix, we systematically measured the loudness of air leak sounds across a matrix with step size 1 cm. Starting from the upper left region of the lung, we recorded sound for 5 seconds and calculated average loudness. The microphone was then advanced to an adjacent square (e.g., 1 cm to the right), and sound acquisition and loudness quantification were repeated to obtain a heatmap showing distribution of the normalized sound intensity, ranging between 0 (no sound) and 1.00 (maximum sound), across the whole lung. To establish the baseline breathing sound intensity matrix, analysis was first performed in uninjured ventilated lungs, where all sound intensity scores ranged 0.19-0.20. Sound intensity analysis was repeated in lungs punctured with an 18-gauge needle. In the breathing sound intensity matrix of lungs with air leak, sound intensity strongly correlated with spatial proximity to the air leak site. Specifically, sound intensity in lungs with air leak ranged 0.18-1.00, where intensity of 1.00 corresponded to the precise location of air leak. This method consistently resulted in precise localization of air leak sites within 1 cm (FIGS. 41, 42).

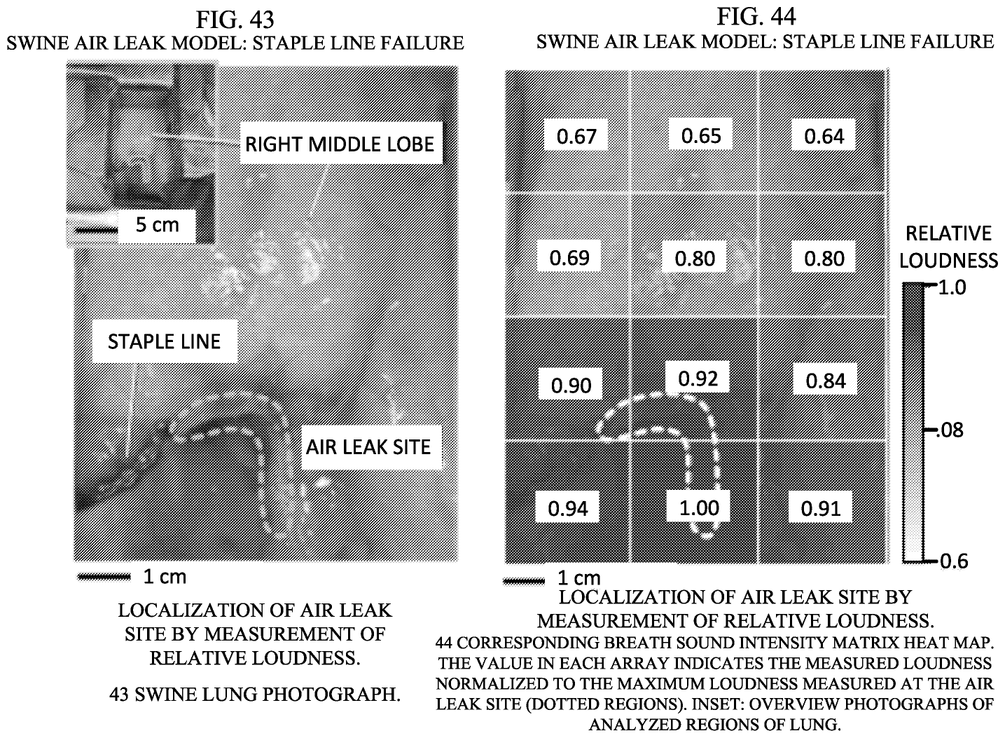

We similarly assessed the ability of our system to locate the site of air leak using sound intensity measurement in human-sized swine lungs. We performed right middle lobe resection and measured sound loudness at 12 points prior to induction of air leak. To demonstrate sensitivity, we performed localization analysis on a mild air leak, which was achieved by removing staples to obtain a 15 mL/breath of air leak. Sound intensity measurements were repeated at the same 12 locations following induction of mild air leak. Normalized sound intensity (range: 0.65-1.00) was greater near the region immediately surrounding the site of air leak (within ˜2 cm of leak site; FIGS. 43, 44), whereas sound intensity (range: 0.37-0.40) did not differ regionally in the lung without air leak injury.

Elimination of heartbeat sound via noise canceling. During air leak sound acquisition and analysis in clinical settings, confounding sound signals originating from background noise (e.g., equipment, other auscultative sounds) can contaminate the air leak sound signals. In particular, while the intensity of background noise can be reduced during measurements, sounds generated by the heart could interfere with measurements due to proximity to the lung. To address this potential issue, we investigated if air leak sound could be detected and isolated in the presence of heart sounds. We generated a sound mixture by adding air leak sound signals collected from swine with staple-line failure (FIG. 38) and heart sounds obtained from a healthy human. Spectral analysis of the sounds revealed that the frequency of human heart sounds ranged between 20 and 400 Hz where each pulse lasted approximately 0.13 s. This substantial difference in frequency range between heart sounds (<400 Hz) and pulmonary air leak sounds (<3,000 Hz) allowed us to effectively attenuate the heartbeat noise using high-pass filtering to extract the air leak signal.

Auscultation of heart and lungs, typically with a stethoscope, has been used for cardiopulmonary evaluation and diagnosis for over a century, but remains reliant on the subjective experience of the care provider. Leveraging the established principle that respiratory sounds can offer diagnostic insight, we developed an objective, quantitative sound analysis methodology that can discern distinct sound signatures of pulmonary air leaks based on key parameters such as frequency distribution and loudness level (FIGS. 16-17). We evaluated air leaks in both small and large animal models to demonstrate the high sensitivity and clinical relevance of this methodology, and determined: (i) air leaks generate discernable sounds that contain harmonic series; (ii) air leak sound properties correlate with volume of air loss, and thus leak severity (FIGS. 21-40); and (iii) air leak loudness correlates with distance from the leak, enabling in situ identification of leak site (FIG. 41). To maximize clinical relevance, we investigated pulmonary air leaks in a swine model due to the anatomical and respiratory (e.g., airway pressure, compliance, tidal volume) similarities to human lungs. To emulate a realistic hospital setting with noisy environment, all studies were conducted in an operating room with standard medical equipment and background noises.

In pulmonary air leaks, air escapes the lung through a defect in a bronchial staple line or the visceral pleura due to a pressure gradient between the alveoli and thorax. If sufficient kinetic energy is transferred from the airflow to surrounding pleural tissue, the tissue oscillates at its resonance frequency which can generate audible sounds (similar to vocal cord vibration during phonation) (FIG. 45). Our results obtained from both small and large lungs consistently showed that sounds generated due to air leak are primarily dependent on the scale of the driving force (i.e., the pressure difference between inside and outside the lungs, AP) and the pleural defect geometry (size, shape), which together modulate the resulting sound loudness, pitch, and spectral density (FIGS. 19, 20, 46, 47, 51, 52, 48, 49 and 50). We applied signal processing algorithms, including noise filtering, to isolate air leak sound signatures for analysis, as envisioned clinical use of this methodology would require filtration of ambient sounds from the patient's heart, medical equipment, and other background noise. This study applied a high-pass filter (cutoff frequency, fc=500 Hz) to attenuate recorded heart sounds for analysis of air leak in an open thoracotomy. By tuning algorithm parameters such as attenuation rate and cutoff frequency of the filter, we detected distinct air leak sound frequency patterns and spectral density profiles, and confirmed that air leak sounds could be isolated from a sound mixture using a high-pass filtering algorithm. While band pass filters and spectral analysis have previously been applied to process heart and lung sounds, our methodology is the first designed to quantitate and characterize pulmonary air leak sounds.

Notably, in both rat and swine models, we discovered that air leak sounds contain harmonic series, in which each frequency is an integer multiple of the fundamental frequency of the oscillating pleural tissue (FIGS. 46, 47, 51 and 52). To our knowledge, this is the first report of harmonic series as a feature of pulmonary air leak. We also determined a statistically significant inverse correlation between sound power and band frequency in both rat and swine (FIG. 30, FIG. 39, FIG. 40, and FIGS. 48-50). The correlation between frequency and relative intensity of bands in air leak harmonic series, as well as the absolute value of the fundamental frequency and relative intensity of bands in these harmonic series of air leak sounds may correspond to physical and pathological features, such as leak location, geometry, underlying lung disease, and tissue stiffness. The fundamental frequency and relative intensity of bands in these harmonic series of air leak sounds may correspond to physical and pathological features, such as leak location, geometry, underlying lung disease, and tissue stiffness. Although the mechanisms of air leak resolution remain to be elucidated, healing likely depends on anatomical factors, as mechanical strain and ventilation are not uniformly distributed throughout the lung, and also on patient-specific factors such as age, underlying lung disease, co-morbidities, immunocompetency, and need for positive-pressure ventilation. Air leak sound signatures and harmonics may correlate with these variables and be used to better understand and predict air leak healing in an individualized manner.

Prior to beginning this study, we interviewed 66 practicing healthcare providers who treat patients with pulmonary air leak (e.g., surgeons, pulmonologists, emergency medicine, nurses, etc.) to confirm the clinical need for improved methods of detecting, assessing, and treating pulmonary air leak. This study was motivated by the fact that existing modalities for detecting air leaks do not allow for precise assessment or localization, and established air leak scoring systems, which are based on nominal categories and analog graduations, are not widely used due to high inter-observer variability. Methods for intraoperative assessment of air leaks rely on submerging the lung in water or exogenous surfactant (i.e., Yang's bubble solution) and visualizing air bubbles that form at the leak site. Results of these air leak tests can be difficult to assess, particularly when direct visualization is difficult, including in peripheral or posterior lung regions or during thoracoscopic (VATS) procedures. Additionally, these methods are qualitative and subjective. Digital chest tube drainage systems attempt to quantitate air leaks, however, they are limited by high cost and variable results. No current modality enables precise localization of air leak sites. Diagnostic modalities such as auscultation, X-ray, computed tomography (CT) scan, and ultrasound can only detect the presence of pneumothorax, and offer minimal or no information about air leak location or severity-a gap in diagnostic capabilities that significantly limits air leak treatment. Here we demonstrate precise localization of air leak site within 1 cm. Finer resolution may be achievable with a smaller microphone or decreased step size in sound intensity matrix measurements. Existing adjunct interventions (e.g., applied suction, pleurodesis, polymer sealant), are inconsistently used due to the lack of objective metrics for leak assessment, difficulty predicting which patients may benefit, and inability to target delivered treatments to the injured region. As a result, clinical management of air leak is often conservative, relying on continued chest tube drainage of air and fluid until the leak heals on its own. In comparison, the proposed sound-guided analysis would offer an objective, quantitative methodology for assessing air leak severity and location which can aid in development of air leak management strategies, enable targeted delivery of adjunct therapeutics, and predict which patients may require additional interventions such as pleurodesis or re-operation.

While findings of this proof-of-concept study demonstrate potential for eventual translation, we acknowledge several limitations and identify future research directions to further strengthen clinical utility of this methodology. Air leaks were only evaluated in the right lung of healthy swine during positive-pressure ventilation. Future studies should assess how air leak sounds differ across lung regions and pathologies, and determine if similar air leak sound signals can be detected in lungs during normal ventilation without mechanical support. Effects of different ventilatory conditions (e.g., peak inspiratory pressure, positive end-expiratory pressure, tidal volumes) on air leak sound intensity and frequency distribution can be investigated to provide comprehensive explanation of the correlation between ventilation regimes and acoustic signatures of air leak. During sound recording from swine lungs, microphone positions were manually manipulated and maintained 1 cm from the surface of the lung. Because measured sound intensity can be influenced by the location of the microphone with respect to measurement site (e.g., distance between microphone and lung surface), establishment of optimized algorithms, and standard operating protocols, and precise upper and lower sound detection limits could further reduce errors and variability between measurements. Detection limits of our methodology would be influenced by aerodynamic interactions between the air and the surrounding tissue at the leak site, sensitivity of the sound acquisition instruments (e.g., microphone, signal amplifier), and the quality of the recordings and noise cancellation methods. Lastly, because all air leak sounds were investigated intraoperatively during non-survival studies with open thoracotomy, changes in air leak sound signatures over extended periods of time and transthoracic air leak sounds were not evaluated. Longitudinal studies can further elucidate correlations between air leak sound properties and post-operative healing within the closed chest.

Based on our finding that air leaks generate unique, quantifiable sound signatures, we envision that this sound analysis methodology could be adapted for use in: (i) open chest surgery, (ii) minimally invasive surgery (VATS), or (iii) noninvasively through closed chest. The application that we explicitly demonstrate in this study is the use of our device in (i) open chest surgery (i.e., thoracotomy). While the water submersion test is used for intraoperative assessment of pulmonary air leak, it can be challenging to visualize peripheral or posterior lung regions and many air leaks may be missed. Our system overcomes this challenge because it can detect air flow from the leak without visualization of the leak site. Our method could be easily adapted for use in (ii) VATS procedures. Standard thoracoscopic ports are 5-12 mm, and the microphone used in this study was 5 mm in diameter. Thus, a smaller microphone may be required to accommodate all surgical instruments that need to pass through the port. The application in VATS is particularly compelling as methods for detecting pulmonary air leak intraoperatively, such as the water submersion test or Yang's bubble solution test, are challenging to perform during VATS procedures. For lung cancer resection in the United States alone, an estimated 66,000 patients undergo open lung surgery each year and an additional 54,000 undergo VATS procedures. Therefore, a significant patient population could benefit from these applications.

The ability to apply quantitative sound analysis in a non-invasive manner through a closed chest presents the opportunity to benefit the greatest number of patients. Globally, 20 million patients receive positive pressure mechanical ventilation each year, and are at increased risk for developing pulmonary air leaks due to barotrauma and underlying lung disease. As pulmonary air leak can rapidly progress to a potentially-fatal tension pneumothorax, rapid detection of air leak is imperative. We envision a modified configuration of our sound analysis system in which wearable sensors are placed on the chest to continuously monitor pulmonary sounds to rapidly detect pulmonary air leak. We recognize several challenges toward application of our system in a closed chest due to transmission characteristics of the intervening chest wall, including, differential coupling, sound attenuation, and spectral distortion. To address these challenges and enable accurate, non-invasive analysis of air leak sounds, we propose incorporation of: (i) accelerometer contact microphone array on the chest; (ii) high pass filter and adaptive filtering; and (iii) triangulation for accurate localization. While the magnitude of the sound signal would be lower in the closed chest, prior work has demonstrated the utility of contact accelerometers for precise, non-invasive analysis of cardiopulmonary sounds of low magnitudes, and could be extended to use in our system. Whereas air coupled microphones are highly sensitive to environmental noises, the incorporation of multiple accelerometers onto the chest wall could enable the use of signal processing techniques including decomposition, independent components analysis, and modulation filtering to allow for precise analysis of sound signals originating from air leak.

A goal of this proof-of-concept study was to demonstrate that pulmonary air leaks generate unique, quantifiable sound signals. The ability of this method to precisely assess and localize pulmonary air leaks through non-invasive acoustic signal analysis through the chest wall remains to be studied. Future device iterations will focus on modifying this system and signatures. For example, we envision that this sound analysis methodology could be adapted for use in: (i) a closed chest; (ii) video-assisted thoracic surgery (VATS) or robot-assisted surgery; (iii) minimally invasive thoracoscopy with steerable robotic device; or (iv) critical care or outpatient settings in the form of a wearable microphone sensor array. In particular, the ability to precisely locate air leaks in a real-time and minimally invasive manner could enable locally targeted and patient-specific administration of therapeutics, such as a polymer-based lung sealant to expedite air leak treatment and improve outcomes. Similar sound detection and analysis methodologies could also be developed for diagnostic evaluations of other organs, such as heart and intestine, where sounds can be evaluated to quantify injury or disease states.

Materials and Methods. The sound acquisition and analysis system comprised a miniature microphone (diameter: 5.6 mm; length: 3 mm), motorized stage to position the microphone, signal amplifier, audio interface, and custom-written MATLAB codes (FIG. 16 and FIG. 18, Notably, standard thoracoscopic ports have inner diameters up to 12 mm, which could allow insertion of the mini-microphone for minimally invasive sound analysis in situ. Sound signals obtained with the microphone passed through the amplifier and audio interface to the computer for sound processing and quantification using custom-written MATLAB scripts, which removed ambient noise, amplified signals, and quantified acquired sounds to predict severity and determine air leak location (FIG. 17). To validate this methodology, we applied quantitative sound analysis in rat and swine models of pulmonary air leak and determined specific acoustic signatures associated with air leaks under different injury states and ventilatory conditions. A rat model of pulmonary air leak in open thoracotomy was established by focal puncture with 16-gauge and 18-gauge needle. Swine model of pulmonary air leak in open thoracotomy was established by wedge resection of right middle lobe with Endo GIA™, Covidien surgical stapler and 60 mm purple load staples, followed by removal of up to 30% of fired staples to induce air leak. Sound signal originating from rat and swine pulmonary air leaks were recorded with Sony ECM 77B and analyzed using custom-written MATLAB codes. All animal procedures were approved by and conducted in accordance with the Institutional Animal Care and Use Committee of Stevens Institute of Technology and Columbia University.

Challenges remain towards individualized management of air leaks with adjunct interventions such as applied suction and pleurodesis. A common problem with air leak patients supported with positive pressure ventilation is optimizing chest tube drainage using suction. While suction can evacuate air to effectively prevent pneumothorax, suction can also negatively impact minute ventilation by creating a positive pressure inflow on a ventilator that is immediately suctioned through a negative pressure chest tube. This prevents the patient from ventilating their lungs, which can lead to hypoxemia, hypercapnia, atelectasis, and consolidation. Due to difficulties in accurate assessment of leak severity, challenges remain toward optimizing suction protocols catered to individual patients.

To accelerate air leak resolution, some providers will perform pleurodesis, in which a second injury is induced on the inside of the chest wall such that scar tissue forms to adhere the lung pleura to the inside of the chest wall to seal the leak and prevent lung collapse. However, difficulties assessing air leak severity make it challenging to determine which patients would require pleurodesis and which patients may have air leaks that will resolve without further intervention. Due to the lack of objective criteria, the decision to administer pleurodesis is often based on physician or institutional preference. Some patients who receive pleurodesis may have air leaks that would otherwise resolve on their own. For these patients, the induction of a second tissue injury may be unnecessary.

Patients who develop air leak often have underlying lung disease, such as chronic obstructive pulmonary disease (COPD), interstitial lung disease (ILD), cystic fibrosis (CF), or acute respiratory distress syndrome (ARDS) secondary to lung injury including COVID-19. Depending on underlying pathology, ventilation strategy can differ dramatically among patients. For example, management of COVID-19 and ARDS patients relies on ventilation with low tidal volume. In order to extract meaningful information from air leak sound signatures, standardized ventilation and measurement protocols must be developed. For example, the impact of positive end expiratory pressure (PEEP), respiratory rate, and ventilatory mode should be considered. Further investigation is needed to optimize ventilatory management for sound analysis across patient populations.

Various pathologies can contribute to changes in lung tissue stiffness (e.g., emphysema, pulmonary fibrosis). Tissue stiffness likely impacts sound signatures because stiffer tissue will oscillate differently than more compliant tissue. Additionally, patients with different pathologies may be more susceptible to certain tissue defect geometries that lead to different air leak ‘densities’. COPD patients may be prone to diffuse air leaks that are distributed evenly across long staple lines, whereas ILD patients may have more focal air leaks at the site of an individual staple. Correlating underlying pathology, tissue stiffness, and air leak sound profile could enable precision diagnosis in the future.

### Materials and Methods

Briefly, a rat model of pulmonary air leak was established by focal puncture with 16-gauge and 18-gauge needle. Swine model of pulmonary air leak was established by wedge resection of right middle lobe with Endo GIA™, Covidien surgical stapler and 60 mm purple load staples, followed by removal of up to 30% of fired staples to induce air leak. Sound signal originating from rat and swine pulmonary air leaks were recorded and analyzed using custom-written MATLAB codes. All animal procedures were approved by and conducted in accordance with the Institutional Animal Care and Use Committee of Stevens Institute of Technology and Columbia University.

Rat model of pulmonary air leak. Sprague-Dawley rats (n=6, equal numbers of male and female animals, 200-250 g; SAS SD Rats, Charles River Laboratories) were used for the rat model of pulmonary air leak. All procedures were performed in accordance with the animal welfare guidelines and regulations of the Institute for Animal Care and Use Committee (IACUC) at Stevens Institute of Technology and Columbia University. Rats were euthanized with 5% isoflurane (VED1350, Penn Veterinary Supply) for 30 minutes using a vaporizer (SomnoSuite, Kent Scientific). An endotracheal cannula (73-2727, Harvard Apparatus) was inserted into the trachea and secured using a 6-0 polypropylene suture. Rats were ventilated using a small animal ventilator (PhysioSuite, Kent Scientific; or Inspira, Harvard Apparatus) with tidal volume of 6 mL/kg at a rate of 70 breaths per minute (bpm). A sternotomy was performed using surgical scalpel and scissors to open the chest. Pulmonary air leak was induced by focal puncture of the visceral pleura of the lung (penetration depth: ˜3 mm) using an 18-gauge (diameter: 1.27 mm) or 16-gauge (diameter: 1.65 mm) needle. Air leak was confirmed by submerging the lung in phosphate-buffered saline (PBS) solution and directly visualizing air bubbles generated at the needle insertion site. Air leak sounds were recorded using our custom-built sound recording and analysis system. All experiments were repeated at least three times.

Rat lung pressure-volume measurements. To monitor the pressure-volume (P-V) relation of the rat lungs, a sensing module was constructed and connected to the ventilator and endotracheal cannula via a three-way stopcock (BD). The module consisted of a pressure sensor (pressure range: ±7 kPa; MPXV7007GC6U, NXP), airflow sensor (flow range: ±750 cm³/min, HAFBLF0200CAAX5, Honeywell), digital acquisition device (DAQ, Arduino Uno), and a computer (XPS 8940, Dell). Pressure and volume data were transferred to the computer via DAQ, then processed and plotted using custom-written MATLAB code (MathWorks). Prior to the measurements, pressure and flow sensors were calibrated according to manufacturer protocol.

Swine model of pulmonary air leak. Yorkshire swine (n=4, female, 35-45 kg, Animal Biotech Industries) were sedated with Telazol (5 mg/kg), anesthetized with 5% isoflurane, intubated with endotracheal tube (7.5 mm), and placed in the left lateral position to access the right lung. Thoracotomy was performed in sterile fashion with a curvilinear incision along the fifth intercostal space. Subcutaneous tissue, muscle, and parietal pleura were dissected, and a rib spreader was used to expose the right middle lobe. A wedge resection of the right middle lobe was performed using a surgical stapler with 60 mm (purple) staple loads (Endo GIA™, Covidien) to remove ˜15 g of lung tissue. To induce air leak that mimics staple-line failure, up to 30% of the staples were removed using forceps and scalpel until tidal volume decreased from baseline by at least 25%. To confirm presence of air leak, the right middle lobe was submerged in normal saline to visualize bubbles. Radiopaque dye (Omnipaque, GE) was delivered using 3.8-mm flexible video bronchoscope (aScope 3; Ambu) to the right lingular bronchus and dye leakage was visualized using portable X-ray unit (PXP-16HF; United Radiology Systems) at 2.2 mAs and 90 kVp. Air leak sounds were recorded in situ with the sound analysis system. All measurements were repeated at least three times. The study received approval from the Institutional Animal Care and Use Committee (IACUC) at Columbia University. All animal care and procedures were conducted in accordance with the US National Research Council's Guide for the Care and Use of Laboratory Animals, 8th edn.

Air leak sound recording system and signal acquisition. A sound measurement platform was constructed by integrating a sound recorder/analyzer network and motorized manipulator. The sound recorder/analyzer consisted of a microphone (Sony ECM-77B, Sony), preamplifier (B12A, Black Lion Audio), audio interface (Fireface 802, RME), and computer (XPS 8940, Dell). Sound signals acquired via the microphone were fed into the preamplifier input channel with maximum gain of 70 dB. Amplified signals were supplied to the audio interface that was connected to the computer via USB cable. During sound measurements in rat lungs, the microphone was mounted on a motorized XYZ manipulator (MiniMill, OpenBuilds) for positioning of the microphone with respect to the measurement site (i.e., air leak site). The motorized manipulator positioned the microphone with submillimeter resolution in three-dimensional space using three integrated motors (total 3 motors; Nema 23 Stepper motor) controlled via Machine Interface Controller software (OpenBuilds). During sound measurements in swine lungs, a human operator positioned the microphone manually. The distance between the microphone tip and operator hand was maintained at 5 cm for all measurements to minimize variation. To maximize signal acquisition and minimize ambient noise, the microphone was positioned perpendicular to the pleural surface 1 cm from the lung pleural surface for all measurements in rat and swine. Audio signals acquired from intact lungs (control) or lungs with air leak were processed for sound analysis and quantification in real time or stored in WAV file format for subsequent sound analysis using custom-written MATLAB codes or open-source audio analysis software (Audacity or Sonic Visualizer). For accurate signal quantification, sound signals were acquired for 5 or 30 seconds (depending on the experiment) through one audio channel with 44.1-kHz sampling rate and 16-bit resolution.

Analysis and visualization of sound spectrogram. To investigate acoustic characteristics of the acquired air leak sounds, spectrograms of the sounds were obtained by extracting and plotting amplitude and frequency distributions of the recorded sound signals against time. Frequency content of the sound waveform that varied with time was computed via the Short-Term Fourier Transform (STFT) of the input audio signals (1, 2). Briefly, STFT can be expressed as:

$$X [ n , k ] = {\sum }_{m = - ∞}^{∞} x [ m ] w [ n - m ] {e}^{- j 2 π \frac{mk}{N}} \qquad [ 1 ]$$

where x[m] is input signal at time m, w[n−m] is length of window, X[n, k] is the Discrete Fourier Transform (DFT) of windowed data at reference time n and frequency k with an integer N. Distribution of frequencies contained in the audio signal was visualized using MATLAB or Audacity in the form of spectrograph which is an intensity plot of STFT magnitude over time and can be obtained by |X[n, k]|². Further, ambient sounds contained in audio signals were removed through filtering (3, 4). For example, to eliminate low-frequency noise, high-pass filter was applied to attenuate sound signals below a cutoff frequency (f_{c}), such as heart sounds (f_{c}: ˜500 Hz).

Quantification of sound pressure level. Pressure level of sounds was quantified by measuring A-weighted sound pressure level (SPL) using SPL meter function implemented in MATLAB. Frequency spectra of acquired sounds were estimated via the Discrete Fast Fourier Transform (DFT) with A-weighting filter. Time-weighted sound level, defined as the ratio of the time-weighted root mean squared sound pressure to the reference sound pressure, was determined. Specifically, DFT of acquired sounds was obtained as follows:

$$X [ k ] = {\sum }_{N = 0}^{N - 1} x [ n ] {e}^{- j 2 π \frac{nk}{N}} \qquad [ 2 ]$$

where x[n] is a discrete input signal with N samples and X[k] is the DFT of the input signal. Notably, DFT provides information about the frequency distribution of input signals with no temporal resolution while STFT described above provides both temporal and frequency resolution. The A− weighting filter (H_{A}(f)) is defined as:

$${H}_{A} ( f ) = \frac{{( 2 π {f}_{4} )}^{2} \cdot {f}^{4}}{{( f + 2 π {f}_{1} )}^{2} \cdot ( f + 2 π {f}_{2} ) \cdot ( f + 2 π {f}_{3} ) \cdot {( f + 2 π {f}_{4} )}^{2}} \qquad [ 3 ]$$

where f₁=20.6 Hz, f₂=107.7 Hz, f₃=737.9 Hz, and f₄=12194.0 Hz. The weight functions and values are specified in the International Electrotechnical Commission (IEC) 61672 which is the current international standard that specifies sound level meter functionality and performance. The A-weighted DFT samples (X_{A}[k]) are then calculated from equations 2 and 3 as:

$${X}_{A} [ k ] = {H}_{A} ( f ) X [ k ] \qquad [ 4 ]$$

Finally, time-weighted sound level (Lₜ), that is equivalent to the sound pressure level (SPL) reported in the manuscript, is determined as:

$${L}_{t} \vert = 10 {log}_{10} \{ \frac{h ( {y}^{2} )}{{P}_{0}^{2}} \} / \qquad [ 5 ]$$

where y is the output of the A-weighting filter, P₀ is a reference pressure defined as the lowest threshold of human hearing (20 μPa), and h(y²) represents the convolution of y² with a filter of impulse response (1/τ)e^{-y/τ} corresponding to a lowpass filter of the form of:

$$H ( s ) = \frac{1 / τ}{s + 1 / τ} \qquad [ 6 ]$$

The Laplace transfer function H(s) was converted into its discrete equivalent H(z) using the impulse invariant transform:

$$H ( z ) = \frac{1 / ( τ \times \text{?} )}{1 - \text{?}} \qquad [ 7 ]$$

$$\text{?} \text{indicates text missing or illegible when filed}$$

where fₛ is the audio sample rate and τ is the time-weighting coefficient (i.e., τ=0.125 for fast time weighting, τ=1 for slow time weighting). In this study, all fₛ was 44.1 KHz and τ used for all sound level evaluation was 0.125. Calculated values of the time-weighted level of A-weighted sound pressure measured were quantified in “A-weighted decibels” (dBA).
Assessment of correlation between the airway pressure and sound pressure level. To quantify association between airway pressure (Pairway) and A-weighted sound pressure level (SPL) acquired from both rat and swing lungs with air leak, we performed the correlation analysis using the time series of the measured data. To do this, all datasets were normalized via the following equation:

$${x}^{′} = \frac{( x - {x}_{min} )}{( {x}_{max} - {x}_{min} )} \qquad [ 8 ]$$

where x′ is the normalized data, x is the original data, xmin is the minimum value in the data set, and xmax is the maximum value in the data set. Then, Pearson correlation coefficients (p) for rat and swine were calculated to determine correlation between the sound intensity and airway pressure (i.e., Pairway, SPL)

$$ρ ( A , B ) = \frac{1}{N - 1} {\sum }_{i = 1}^{N} ( \frac{{A}_{i} - {μ}_{A}}{{σ}_{A}} ) ( \frac{{B}_{i} - {μ}_{B}}{{σ}_{B}} ) \qquad [ 9 ]$$

where μA and σA are the mean and standard deviation of a data set A, respectively, and μB and σB are the mean and standard deviation of a data set B.

Generation of sound intensity map. The heatmap of the sound intensity for rat and swine lungs were obtained by measuring an average SPL value at different locations across the surface of the lung. Acoustic scanning was performed by positioning the microphone 1 cm from the lung pleural surface at the point of interest (i.e., upper left corner). At each point of interest, sounds were recorded for 5 seconds and average sound pressure level was calculated using MATLAB immediately after the sound recording. Once measurement was completed at a point, the microphone was moved by 1 cm for rat lungs and 2 cm for swine lung to an adjacent point for sound intensity quantification across a specified area of the lung. The total acoustic scan area for rat lungs and swine lungs were 5 cm×5 cm and 6 cm×8 cm, respectively. The average SPL values obtained within the scanned areas were normalized against the maximum SPL value recorded from each lung, which was consistently obtained just above the air leak site. This normalization resulted in the SPL values ranging between 0 and 1 that were used to create sound intensity maps. In the maps, the maximum SPL is indicated by “red” and the minimum SPL is indicated by “white” while intermediate values are interpolated between these two colors. To facilitate visualization of the sound intensity at the measurement area, the sound intensity map and a photograph of corresponding lung region were overlaid.

Statistical tests. All data were obtained from experiments repeated at least three times. All data analyses were conducted in Microsoft Excel and R. Results are reported as the mean and standard deviation of measured values. One-way analysis of variance (ANOVA) was used to determine statistically significant differences between groups, with p<0.05 considered significant.

It will be understood that the embodiments described hereinabove are merely exemplary and that a person skilled in the art may make many variations and modifications without departing from the spirit and scope of the present invention. All such variations and modifications are intended to be included within the scope of the invention as defined in the appended claims.

1-25. (canceled)

26. A non-invasive apparatus for detecting pulmonary air leaks of a human patient, comprising:
a garment having a plurality of sensors adapted to detect acoustic signals produced by a pulmonary air leak internal to a wearer of the garment;
an amplifier operatively engaged to the plurality of sensors, the amplifier configured to amplify the acoustic signals detected by the plurality of sensors and to thereby produce amplified acoustic signals;
an acoustic analyzer adapted to receive the amplified acoustic signals produced by the amplifier, the acoustic analyzer being programmed with sound localization algorithms to determine the location of an air leak represented by the acoustic signals detected by the plurality of sensors; and
a therapeutic delivery system operatively coupled to the garment, the therapeutic delivery system including a steerable therapeutic applicator having a longitudinal body with a proximal end, a distal end and a length therebetween, the therapeutic applicator further including a plurality of channels extending along at least a portion of the length of the longitudinal body, a first channel of the plurality of channels being configured to receive and deliver a therapeutic to the location of an air leak represented by the acoustic signals detected by the plurality of sensors.

27. The apparatus of claim 26, wherein the plurality of sensors is arranged in an array.

28. The apparatus of claim 27, wherein the plurality of sensors is arranged in a 3×3 array or a 6×6 array on the posterior side and/or the anterior side of the garment.

30. The apparatus of claim 26, wherein the acoustic analyzer is a computer programmed to use the sound localization algorithms to assess differences in time-arrival and intensity of the acoustic signals detected by the plurality of sensors.

31. The apparatus of claim 30, wherein the computer is programmed with noise filtering algorithms to isolate acoustic signals unique to an air leak.

32. The apparatus of claim 31, wherein the computer is programmed to filter out ambient noise, as well as heartbeat and other background noises of a wearer of the garment.

33. The apparatus of claim 26, wherein the plurality of channels of the therapeutic applicator further includes a second channel configured to receive a microphone for air leak sound recording and a third channel configured to receive an optical fiber for optical guidance of the therapeutic applicator inside a wearer's chest cavity.

34. The apparatus of claim 33, wherein the first channel has a diameter of 2 mm, the second channel has a diameter of 1 mm and the third channel has a diameter of 1 mm.

35. The apparatus of claim 33, wherein the therapeutic applicator is steered by visual servoing based upon information collected via the optical fiber received in the third channel.

36. The apparatus of claim 26, wherein the therapeutic delivery system further includes a motor and motor controller operatively engaged to the therapeutic applicator.

37. The apparatus of claim 26, wherein the therapeutic applicator includes a plurality of pulling wires along the length of the body thereof, the therapeutic applicator being steerable by at least one motor attached to the pulling wires.

38. The apparatus of claim 26, wherein the therapeutic applicator is configured to target and deliver therapeutic by optoacoustic guidance.

39. The apparatus of claim 26, wherein the first channel of the therapeutic applicator is preloaded with therapeutic for air leak repair.

40. The apparatus of claim 39, wherein the therapeutic is a biosealant hydrogel composition adapted to seal an air leak represented by the acoustic signals detected by the plurality of sensors.

41. The apparatus of claim 26, wherein the plurality of channels comprises flexible polymeric conduits with guidewire disks attached to an outer surface of a distal end of each of the conduits.

42. The apparatus of claim 26, wherein the therapeutic applicator is steered manually or using computer vision guidance.

43. A method for treating pulmonary air leaks of a human patient, the method comprises the steps of:
inserting a therapeutic applicator into a patient's chest;
determining a location of a pulmonary air leak using a microphone operatively coupled to the therapeutic applicator;
steering the therapeutic applicator to the determined location of the pulmonary air leak;
visualizing the determined location of the pulmonary air leak using an optical fiber imaging probe operatively coupled to the therapeutic applicator; and
delivering a therapeutic to the determined location of the pulmonary air leak.

44. The method of claim 43, wherein the therapeutic applicator is operative engaged to a garment adapted to be worn by the patient.

45. The method of claim 44, wherein the garment is in the form of a vest or chest strap.
